SEQUENCE LISTING (14 sequences)

SEQ ID NO: 1 - DNA, 1431 bp, Mus musculus
  source 1..1431: mol_type = other DNA
atggccgggcgcggaggacgggtgctgctggcgctgtgtgccgcgctggtggccggcgggtggctgctgacggctgaagcccaggagcccggggcgccagcggctggcatgaggcgccgccggcggctccagcaagaggacggcatctccttcgagtaccaccgctatccagagctgcgcgaggcgctggtgtccgtatggctgcagtgcaccgccatcagcagaatctacacagtggggcgcagcttcgagggccgggagctcctggtcatcgagctgtctgacaaccccggggtccatgagccgggtgaacctgaatttaaatacattgggaacatgcatggtaatgaggcggttggacgggaactgcttattttcttggcccagtacctgtgtaacgagtaccagaaaggcaatgagacaattgtcaacctgatccacagcacccgaattcatatcatgccctccttgaaccccgacggctttgagaaagccgcatcgcagcccggcgagctgaaggactggtttgtgggccgcagcaacgcccagggaatagatctgaaccgtaacttcccagacctggacaggatcgtgtatgttaatgagaaagaaggcggtcctaacaatcacctgctgaagaatctgaagaaaattgtggaccaaaattcaaagcttgcccccgagaccaaggctgtcattcactggatcatggacattccatttgtgctttctgccaatctgcacggaggagaccttgtggctaattacccatatgatgagacacggagcggtactgctcacgaatacagttcctgccctgatgacgcaattttccaaagcttggctcgcgcgtactcttctttcaacccagtcatgtctgaccccaatcgacctccctgtcgcaagaatgacgatgacagcagctttgtagatggaacgaccaatggtggtgcatggtacagcgtccccggtggaatgcaagacttcaattacctgagcagcaactgcttcgagatcactgtggagcttagctgtgagaagttcccaccggaagagactctcaaaagctactgggaagataacaaaaactccctcatcagctacctggagcagatacaccgaggtgttaaagggtttgtccgtgaccttcagggtaacccgattgccaacgcaaccatctctgtggacgggatagaccatgatgtcacctcggctaaggatggggattactggcgattgcttgctcctggaaactataaacttacagcctccgctcctggctacctggcaatcacaaagaaagtggcagttccttttagccctgctgttggggtggactttgagcttgagtctttctctgaaaggaaggaggaggagaaggaagaattgatggagtggtggaaaatgatgtcagaaactttgaatttttaa

SEQ ID NO: 2 - protein, 476 aa, Mus musculus
MAGRGGRVLLALCAALVAGGWLLTAEAQEPGAPAAGMRRRRRLQQEDGISFEYHRYPELREALVSVWLQCTAISRIYTVGRSFEGRELLVIELSDNPGVHEPGEPEFKYIGNMHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNLKKIVDQNSKLAPETKAVIHWIMDIPFVLSANLHGGDLVANYPYDETRSGTAHEYSSCPDDAIFQSLARAYSSFNPVMSDPNRPPCRKNDDDSSFVDGTTNGGAWYSVPGGMQDFNYLSSNCFEITVELSCEKFPPEETLKSYWEDNKNSLISYLEQIHRGVKGFVRDLQGNPIANATISVDGIDHDVTSAKDGDYWRLLAPGNYKLTASAPGYLAITKKVAVPFSPAVGVDFELESFSERKEEEKEELMEWWKMMSETLNF

SEQ ID NO: 3 - DNA, 1431 bp, Homo sapiens
  source 1..1431: mol_type = other DNA
atggccgggcgagggggcagcgcgctgctggctctgtgcggggcactggctgcctgcgggtggctcctgggcgccgaagcccaggagcccggggcgcccgcggcgggcatgaggcggcgccggcggctgcagcaagaggacggcatctccttcgagtaccaccgctaccccgagctgcgcgaggcgctcgtgtccgtgtggctgcagtgcaccgccatcagcaggatttacacggtggggcgcagcttcgagggccgggagctcctggtcatcgagctgtccgacaaccctggcgtccatgagcctggtgagcctgaatttaaatacattgggaatatgcatgggaatgaggctgttggacgagaactgctcattttcttggcccagtacctatgcaacgaataccagaaggggaacgagacaattgtcaacctgatccacagtacccgcattcacatcatgccttccctgaacccagatggctttgagaaggcagcgtctcagcctggtgaactcaaggactggtttgtgggtcgaagcaatgcccagggaatagatctgaaccggaactttccagacctggataggatagtgtacgtgaatgagaaagaaggtggtccaaataatcatctgttgaaaaatatgaagaaaattgtggatcaaaacacaaagcttgctcctgagaccaaggctgtcattcattggattatggatattccttttgtgctttctgccaatctccatggaggagaccttgtggccaattatccatatgatgagacgcggagtggtagtgctcacgaatacagctcctccccagatgacgccattttccaaagcttggcccgggcatactcttctttcaacccggccatgtctgaccccaatcggccaccatgtcgcaagaatgatgatgacagcagctttgtagatggaaccaccaacggtggtgcttggtacagcgtacctggagggatgcaagacttcaattaccttagcagcaactgttttgagatcaccgtggagcttagctgtgagaagttcccacctgaagagactctgaagacctactgggaggataacaaaaactccctcattagctaccttgagcagatacaccgaggagttaaaggatttgtccgagaccttcaaggtaacccaattgcgaatgccaccatctccgtggaaggaatagaccacgatgttacatccgcaaaggatggtgattactggagattgcttatacctggaaactataaacttacagcctcagctccaggctatctggcaataacaaagaaagtggcagttccttacagccctgctgctggggttgattttgaactggagtcattttctgaaaggaaagaagaggagaaggaagaattgatggaatggtggaaaatgatgtcagaaactttaaatttttaa

SEQ ID NO: 4 - protein, 476 aa, Homo sapiens
MAGRGGSALLALCGALAACGWLLGAEAQEPGAPAAGMRRRRRLQQEDGISFEYHRYPELREALVSVWLQCTAISRIYTVGRSFEGRELLVIELSDNPGVHEPGEPEFKYIGNMHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNMKKIVDQNTKLAPETKAVIHWIMDIPFVLSANLHGGDLVANYPYDETRSGSAHEYSSSPDDAIFQSLARAYSSFNPAMSDPNRPPCRKNDDDSSFVDGTTNGGAWYSVPGGMQDFNYLSSNCFEITVELSCEKFPPEETLKTYWEDNKNSLISYLEQIHRGVKGFVRDLQGNPIANATISVEGIDHDVTSAKDGDYWRLLIPGNYKLTASAPGYLAITKKVAVPYSPAAGVDFELESFSERKEEEKEELMEWWKMMSETLNF

SEQ ID NO: 5 - DNA, 1431 bp, Mus musculus
  source 1..1431: mol_type = other DNA
atggccgggcgcggaggacgggtgctgctggcgctgtgtgccgcgctggtggccggcgggtggctgctgacggctgaagcccaggagcccggggcgccagcggctggcatgaggcgccgccggcggctccagcaagaggacggcatctccttcgagtaccaccgctatccagagctgcgcgaggcgctggtgtccgtatggctgcagtgcaccgccatcagcagaatctacacagtggggcgcagcttcgagggccgggagctcctggtcatcgagctgtctgacaaccccggggtccatgagccgggtgaacctgaatttaaatacattgggaacatgcatggtaatgaggcggttggacgggaactgcttattttcttggcccagtacctgtgtaacgagtaccagaaaggcaatgagacaattgtcaacctgatccacagcacccgaattcatatcatgccctccttgaaccccgacggctttgagaaagccgcatcgcagcccggcgagctgaaggactggtttgtgggccgcagcaacgcccagggaatagatctgaaccgtaacttcccagacctggacaggatcgtgtatgttaatgagaaagaaggcggtcctaacaatcacctgctgaagaatctgaagaaaattgtggaccaaaattcaaagcttgcccccgagaccaaggctgtcattcactggatcatggacattccatttgtgctttctgccaatctgcacggaggagaccttgtggctaattacccatatgatgagacacggagcggtactgctcacgaatacagttcctgccctgatgacgcaattttccaaagcttggctcgcgcgtactcttctttcaacccagtcatgtctgaccccaatcgacctccctgtcgcaagaatgacgatgacagcagctttgtagatggaacgaccaatggtggtgcatggtacagcgtccccggtggaatgcaagacttcaattacctgagcagcaactgcttcgagatcactgtgcagcttagctgtgagaagttcccaccggaagagactctcaaaagctactgggaagataacaaaaactccctcatcagctacctggagcagatacaccgaggtgttaaagggtttgtccgtgaccttcagggtaacccgattgccaacgcaaccatctctgtggacgggatagaccatgatgtcacctcggctaaggatggggattactggcgattgcttgctcctggaaactataaacttacagcctccgctcctggctacctggcaatcacaaagaaagtggcagttccttttagccctgctgttggggtggactttgagcttgagtctttctctgaaaggaaggaggaggagaaggaagaattgatggagtggtggaaaatgatgtcagaaactttgaatttttaa

SEQ ID NO: 6 - protein, 476 aa, Mus musculus
MAGRGGRVLLALCAALVAGGWLLTAEAQEPGAPAAGMRRRRRLQQEDGISFEYHRYPELREALVSVWLQCTAISRIYTVGRSFEGRELLVIELSDNPGVHEPGEPEFKYIGNMHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNLKKIVDQNSKLAPETKAVIHWIMDIPFVLSANLHGGDLVANYPYDETRSGTAHEYSSCPDDAIFQSLARAYSSFNPVMSDPNRPPCRKNDDDSSFVDGTTNGGAWYSVPGGMQDFNYLSSNCFEITVQLSCEKFPPEETLKSYWEDNKNSLISYLEQIHRGVKGFVRDLQGNPIANATISVDGIDHDVTSAKDGDYWRLLAPGNYKLTASAPGYLAITKKVAVPFSPAVGVDFELESFSERKEEEKEELMEWWKMMSETLNF

SEQ ID NO: 7 - DNA, 1431 bp, Homo sapiens
  source 1..1431: mol_type = other DNA
atggccgggcgagggggcagcgcgctgctggctctgtgcggggcactggctgcctgcgggtggctcctgggcgccgaagcccaggagcccggggcgcccgcggcgggcatgaggcggcgccggcggctgcagcaagaggacggcatctccttcgagtaccaccgctaccccgagctgcgcgaggcgctcgtgtccgtgtggctgcagtgcaccgccatcagcaggatttacacggtggggcgcagcttcgagggccgggagctcctggtcatcgagctgtccgacaaccctggcgtccatgagcctggtgagcctgaatttaaatacattgggaatatgcatgggaatgaggctgttggacgagaactgctcattttcttggcccagtacctatgcaacgaataccagaaggggaacgagacaattgtcaacctgatccacagtacccgcattcacatcatgccttccctgaacccagatggctttgagaaggcagcgtctcagcctggtgaactcaaggactggtttgtgggtcgaagcaatgcccagggaatagatctgaaccggaactttccagacctggataggatagtgtacgtgaatgagaaagaaggtggtccaaataatcatctgttgaaaaatatgaagaaaattgtggatcaaaacacaaagcttgctcctgagaccaaggctgtcattcattggattatggatattccttttgtgctttctgccaatctccatggaggagaccttgtggccaattatccatatgatgagacgcggagtggtagtgctcacgaatacagctcctccccagatgacgccattttccaaagcttggcccgggcatactcttctttcaacccggccatgtctgaccccaatcggccaccatgtcgcaagaatgatgatgacagcagctttgtagatggaaccaccaacggtggtgcttggtacagcgtacctggagggatgcaagacttcaattaccttagcagcaactgttttgagatcaccgtgcagcttagctgtgagaagttcccacctgaagagactctgaagacctactgggaggataacaaaaactccctcattagctaccttgagcagatacaccgaggagttaaaggatttgtccgagaccttcaaggtaacccaattgcgaatgccaccatctccgtggaaggaatagaccacgatgttacatccgcaaaggatggtgattactggagattgcttatacctggaaactataaacttacagcctcagctccaggctatctggcaataacaaagaaagtggcagttccttacagccctgctgctggggttgattttgaactggagtcattttctgaaaggaaagaagaggagaaggaagaattgatggaatggtggaaaatgatgtcagaaactttaaatttttaa

SEQ ID NO: 8 - protein, 476 aa, Homo sapiens
MAGRGGSALLALCGALAACGWLLGAEAQEPGAPAAGMRRRRRLQQEDGISFEYHRYPELREALVSVWLQCTAISRIYTVGRSFEGRELLVIELSDNPGVHEPGEPEFKYIGNMHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNMKKIVDQNTKLAPETKAVIHWIMDIPFVLSANLHGGDLVANYPYDETRSGSAHEYSSSPDDAIFQSLARAYSSFNPAMSDPNRPPCRKNDDDSSFVDGTTNGGAWYSVPGGMQDFNYLSSNCFQITVELSCEKFPPEETLKTYWEDNKNSLISYLEQIHRGVKGFVRDLQGNPIANATISVEGIDHDVTSAKDGDYWRLLIPGNYKLTASAPGYLAITKKVAVPYSPAAGVDFELESFSERKEEEKEELMEWWKMMSETLNF

SEQ ID NO: 9 - RNA, 1729 nt, Mus musculus
  source 1..1729: mol_type = other RNA
gtccatgagccgggtgaacctgaatttaaatacattgggaacatgcatggtaatgaggcggttggacgggaactgcttattttcttggcccagtacctgtgtaacgagtaccagaaaggcaatgagacaattgtcaacctgatccacagcacccgaattcatatcatgccctccttgaaccccgacggctttgagaaagccgcatcgcagcccggcgagctgaaggactggtttgtgggccgcagcaacgcccagggaatagatctgaaccgtaacttcccagacctggacaggatcgtgtatgttaatgagaaagaaggcggtcctaacaatcacctgctgaagaatctgaagaaaattgtggaccaaaattcaaagcttgcccccgagaccaaggctgtcattcactggatcatggacattccatttgtgctttctgccaatctgcacggaggagaccttgtggctaattacccatatgatgagacacggagcggtactgctcacgaatacagttcctgccctgatgacgcaattttccaaagcttggctcgcgcgtactcttctttcaacccagtcatgtctgaccccaatcgacctccctgtcgcaagaatgacgatgacagcagctttgtagatggaacgaccaatggtggtgcatggtacagcgtccccggtggaatgcaagacttcaattacctgagcagcaactgcttcgagatcactgtggagcttagctgtgagaagttcccaccggaagagactctcaaaagctactgggaagataacaaaaactccctcatcagctacctggagcagatacaccgaggtgttaaagggtttgtccgtgaccttcagggtaacccgattgccaacgcaaccatctctgtggacgggatagaccatgatgtcacctcggctaaggatggggattactggcgattgcttgctcctggaaactataaacttacagcctccgctcctggctacctggcaatcacaaagaaagtggcagttccttttagccctgctgttggggtggactttgagcttgagtctttctctgaaaggaaggaggaggagaaggaagaattgatggagtggtggaaaatgatgtcagaaactttgaatttttaagaaaggcttctaactaattgctttaatctatctatagactgtagtaagatgcaatgtggctcttttcttttaggttgtgtgcagttgatatttaacattgatttatttttgatcatttaagtaatagttagtaatcacgtaaatacacccggacagaaatataatgtctggatctacttcattcttacatcaacattcactttaaaatctatcgaagctcttttaacgtaatgggtgacaatgtcacatgacagatgccatgaagaagtcaaccgatatagcttggatctgtgaaccctgtactgcgagaatcacatagttccatataagttgtccttagtctcttgtgctgattcactgtataagcatgatcctggtaatgcactttggatgggaagaaaatgtacgtgcttttcagaggggctctgaacagaatgaaaacctagttcttgcgtgtactttgaagaatggaattgtattagtcagcctgttaatgccacttcagagtttggggttttgtcttgattgtagattggcccagaattgcattctgatgaataaaggcaaaaaaaaaaaaaaaaaaaaaaaaaaa

SEQ ID NO: 10 - DNA, 1095 bp, Mus musculus
  source 1..1095: mol_type = other DNA
atgcatggtaatgaggcggttggacgggaactgcttattttcttggcccagtacctgtgtaacgagtaccagaaaggcaatgagacaattgtcaacctgatccacagcacccgaattcatatcatgccctccttgaaccccgacggctttgagaaagccgcatcgcagcccggcgagctgaaggactggtttgtgggccgcagcaacgcccagggaatagatctgaaccgtaacttcccagacctggacaggatcgtgtatgttaatgagaaagaaggcggtcctaacaatcacctgctgaagaatctgaagaaaattgtggaccaaaattcaaagcttgcccccgagaccaaggctgtcattcactggatcatggacattccatttgtgctttctgccaatctgcacggaggagaccttgtggctaattacccatatgatgagacacggagcggtactgctcacgaatacagttcctgccctgatgacgcaattttccaaagcttggctcgcgcgtactcttctttcaacccagtcatgtctgaccccaatcgacctccctgtcgcaagaatgacgatgacagcagctttgtagatggaacgaccaatggtggtgcatggtacagcgtccccggtggaatgcaagacttcaattacctgagcagcaactgcttcgagatcactgtggagcttagctgtgagaagttcccaccggaagagactctcaaaagctactgggaagataacaaaaactccctcatcagctacctggagcagatacaccgaggtgttaaagggtttgtccgtgaccttcagggtaacccgattgccaacgcaaccatctctgtggacgggatagaccatgatgtcacctcggctaaggatggggattactggcgattgcttgctcctggaaactataaacttacagcctccgctcctggctacctggcaatcacaaagaaagtggcagttccttttagccctgctgttggggtggactttgagcttgagtctttctctgaaaggaaggaggaggagaaggaagaattgatggagtggtggaaaatgatgtcagaaactttgaatttttaa

SEQ ID NO: 11 - protein, 364 aa, Mus musculus
MHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNLKKIVDQNSKLAPETKAVIHWIMDIPFVLSANLHGGDLVANYPYDETRSGTAHEYSSCPDDAIFQSLARAYSSFNPVMSDPNRPPCRKNDDDSSFVDGTTNGGAWYSVPGGMQDFNYLSSNCFEITVELSCEKFPPEETLKSYWEDNKNSLISYLEQIHRGVKGFVRDLQGNPIANATISVDGIDHDVTSAKDGDYWRLLAPGNYKLTASAPGYLAITKKVAVPFSPAVGVDFELESFSERKEEEKEELMEWWKMMSETLNF

SEQ ID NO: 12 - RNA, 1569 nt, Homo sapiens
  source 1..1569: mol_type = other RNA
cattcagccggggaaggtgaggcgagtagaggctggtgcggaacttgccgccccctgaggcggcgccggcggctgcagcaagaggacggcatctccttcgagtaccaccgctaccccgagctgcgcgaggcgctcgtgtccgtgtggctgcagtgcaccgccatcagcaggatttacacggtggggcgcagcttcgagggccgggagctcctggtcatcgagctgtccgacaaccctggcgtccatgagcctggtgagcctgaatttaaatacattgggaatatgcatgggaatgaggctgttggacgagaactgctcattttcttggcccagtacctatgcaacgaataccagaaggggaacgagacaattgtcaacctgatccacagtacccgcattcacatcatgccttccctgaacccagatggctttgagaaggcagcgtctcagcctggtgaactcaaggactggtttgtgggtcgaagcaatgcccagggaatagatctgaaccggaactttccagacctggataggatagtgtacgtgaatgagaaagaaggtggtccaaataatcatctgttgaaaaatatgaagaaaattgtggatcaaaacacaaagcttgctcctgagaccaaggctgtcattcattggattatggatattccttttgtgctttctgccaatctccatggaggagaccttgtggccaattatccatatgatgagacgcggagtggtagtgctcacgaatacagctcctccccagatgacgccattttccaaagcttggcccgggcatactcttctttcaacccggccatgtctgaccccaatcggccaccatgtcgcaagaatgatgatgacagcagctttgtagatggaaccaccaacggtggtgcttggtacagcgtacctggagggatgcaagacttcaattaccttagcagcaactgttttgagatcaccgtggagcttagctgtgagaagttcccacctgaagagactctgaagacctactgggaggataacaaaaactccctcattagctaccttgagcagatacaccgaggagttaaaggatttgtccgagaccttcaaggtaacccaattgcgaatgccaccatctccgtggaaggaatagaccacgatgttacatccgcaaaggatggtgattactggagattgcttatacctggaaactataaacttacagcctcagctccaggctatctggcaataacaaagaaagtggcagttccttacagccctgctgctggggttgattttgaactggagtcattttctgaaaggaaagaagaggagaaggaagaattgatggaatggtggaaaatgatgtcagaaactttaaatttttaaaaaggcttctagttagctgctttaaatctatctatataatgtagtatgatgtaatgtggtcttttttttagattttgtgcagttaatacttaacattgatttattttttaatcatttaaatattaatcaactttccttaaaataaatagcctcttaggtaaaaaaaaaaaaaaaaaaaaaaaaaaaaaaaaa

SEQ ID NO: 13 - DNA, 1095 bp, Homo sapiens
  source 1..1095: mol_type = other DNA
atgcatgggaatgaggctgttggacgagaactgctcattttcttggcccagtacctatgcaacgaataccagaaggggaacgagacaattgtcaacctgatccacagtacccgcattcacatcatgccttccctgaacccagatggctttgagaaggcagcgtctcagcctggtgaactcaaggactggtttgtgggtcgaagcaatgcccagggaatagatctgaaccggaactttccagacctggataggatagtgtacgtgaatgagaaagaaggtggtccaaataatcatctgttgaaaaatatgaagaaaattgtggatcaaaacacaaagcttgctcctgagaccaaggctgtcattcattggattatggatattccttttgtgctttctgccaatctccatggaggagaccttgtggccaattatccatatgatgagacgcggagtggtagtgctcacgaatacagctcctccccagatgacgccattttccaaagcttggcccgggcatactcttctttcaacccggccatgtctgaccccaatcggccaccatgtcgcaagaatgatgatgacagcagctttgtagatggaaccaccaacggtggtgcttggtacagcgtacctggagggatgcaagacttcaattaccttagcagcaactgttttgagatcaccgtggagcttagctgtgagaagttcccacctgaagagactctgaagacctactgggaggataacaaaaactccctcattagctaccttgagcagatacaccgaggagttaaaggatttgtccgagaccttcaaggtaacccaattgcgaatgccaccatctccgtggaaggaatagaccacgatgttacatccgcaaaggatggtgattactggagattgcttatacctggaaactataaacttacagcctcagctccaggctatctggcaataacaaagaaagtggcagttccttacagccctgctgctggggttgattttgaactggagtcattttctgaaaggaaagaagaggagaaggaagaattgatggaatggtggaaaatgatgtcagaaactttaaatttttaa

SEQ ID NO: 14 - protein, 361 aa, Homo sapiens
MHGNEAVGRELLIFLAQYLCNEYQKGNETIVNLIHSTRIHIMPSLNPDGFEKAASQPGELKDWFVGRSNAQGIDLNRNFPDLDRIVYVNEKEGGPNNHLLKNMKKIVDQNTKLAPETKAVIHWIMDIPFVLSANLHGGDLVANYPYDETRSGSAHEYSSSPDDAIFQSLARAYSSFNPAMSDPNRPPCRKNDDDSSFVDGTTNGGAWYSVPGGMQDFNYLSSNCFEITVELSCEKFPPEETLKTYWEDNKNSLISYLEQIHRGVKGFVRDLQGNPIANATISVEGIDHDVTSAKDGDYWRLLIPGNYKLTASAPGYLAITKKVAVPYSPAAGVDFELESFSERKEEEKEELMEWWKMMSET

(19) United States
(12) Patent Application Publication
(10) Pub. No.: US 2026/0199441 A1
(43) Pub. Date: Jul. 16, 2026

(54) USE OF CARBOXYPEPTIDASE E/NEUROTROPHIC FACTOR-ALPHA1 TO TREAT NEURODEGENERATIVE DISEASE

(71) Applicant: The USA, as represented by the Secretary, Department of Health and Human Services, Bethesda, MD (US)

(72) Inventor: Yoke Peng LOH, Bethesda, MD (US)

(21) Appl. No.: 18/705,869

(22) PCT Filed: Oct. 26, 2022
(86) PCT No.: PCT/US2022/078713

§ 371 Date: Apr. 29, 2024

Related U.S. Application Data
(60) Provisional application No. 63/273,312, filed on Oct. 29, 2021.

(51) Int. Cl. A61K 38/48; A61K 48/00; A61P 25/28; C12N 15/86

(52) U.S. Cl. CPC A61K 38/4813; A61K 48/005; A61P 25/28; C12N 15/86; C12Y 304/1701; C12N 2750/14143

(57) ABSTRACT

The present disclosure relates to use of Carboxypeptidase E (CPE) for treating or preventing onset or progression of a neurodegenerative disease.

### FIELD OF THE INVENTION

The present invention relates to use of Carboxypeptidase E (CPE)/neurotrophic factor-alphal (NF-α1) to treat neurodegenerative diseases.

### SEQUENCE LISTING

The present application contains a Sequence Listing which has been submitted in .XML format via PatentCenter and is hereby incorporated herein by reference in its entirety. Said WIPO Sequence Listing was created on 19 Oct. 2022 is named 060734_740384_SequenceListing.xml and is 26.3 kilobytes in size.

### BACKGROUND OF THE INVENTION

Carboxypeptidase E (CPE) is a prohormone processing enzyme expressed abundantly in the hippocampus of normal animals including humans. CPE was first discovered in 1982 as a member of the M14 metallocarboxypeptidase family in bovine adrenal medulla that functions as a prohormone processing enzyme. CPE cleaves C-terminal basic amino acids from the intermediates generated by proprotein convertases' action on prohormones and pro-neuropeptides, thereby producing bioactive hormones and neuropeptides (Hook et al., 1982; Fricker and Snyder, 1983; Fricker, 1988). In the central nervous system, CPE also functions as a regulated secretory pathway sorting receptor, secretory vesicle transport regulator and mediates synaptic vesicle localization to the active zone for release (Cawley et al., 2012; Ji et al., 2017). Subsequent studies have shown that CPE is a new neurotrophic factor, functioning extracellularly, independent of its enzymatic activity, in the adult and embryonic central nervous system (Cheng et al. 2013, Selveraj et al. 2017, Ji et al., 2017, Xiao et al. 2019). Hence it was given an additional name of Neurotrophic factor-α1 (NF-α1) which better describes its new function. Human mutations of CPE/NF-α1 have been associated with obesity, diabetes, infertility, intellectual disabilities, and Alzheimer's disease (Alsters et al., 2015; Cheng et al 2016b, Dumarz et al. 2021, Bosch et al., 2021).

Alzheimer's disease (AD) is one of the most devastating neurodegenerative disorders that cause dementia and decreased cognitive function. It currently affects 6.2 million Americans in 2021 and projected to reach 12.7 million by 2050 according to an annual report recently released by the Alzheimer's Association (2021 Alzheimer's Disease Facts and Figures). Accumulation of amyloid β-peptide (Aβ) plaque extracellularly and formation of neurofibrillary tangles from hyperphosphorylated tau intracellularly are pathological hallmarks of AD that generally precede the clinical symptoms. Accordingly, there is a need in the art for effective drugs that can prevent or reverse AD.

### SUMMARY OF THE INVENTION

Provided herein is a method of treating a neurodegenerative disease in a subject. The method may comprise administering to the subject a pharmaceutical composition comprising Carboxypeptidase E (CPE) or a polynucleotide encoding CPE.

Also provided herein is a method of preventing onset or progression of a neurodegenerative disease in a subject. The method may comprise administering to the subject a pharmaceutical composition comprising CPE or a polynucleotide encoding CPE.

For any of the methods described herein, the CPE may be a mouse CPE. The mouse CPE may comprise an amino acid sequence that is at least 95% identical to SEQ ID NO:2. One example of a mouse CPE may be a native protein, which may comprise the amino acid sequence as set forth in SEQ ID NO:2. Another example of a mouse CPE may be a mutant CPE. The mutant CPE may be CPE-E342Q, which may comprise the amino acid sequence as set forth in SEQ ID NO:6. Still another example of a mouse CPE may be a N-terminal-truncated variant (CPE-ΔN), which may comprise the amino acid sequence as set forth in SEQ ID NO:11.

Alternatively, for any of the methods described herein, the CPE may be a human CPE. The human CPE may comprise an amino acid sequence that is at least 95% identical to SEQ ID NO:4. One example of a human CPE may be a native protein, which may comprise the amino acid sequence as set forth in SEQ ID NO:4. Another example of a human CPE may be a mutant CPE. The mutant CPE may be CPE-E342Q, which may comprise the amino acid sequence as set forth in SEQ ID NO:8. Still another example of a mouse CPE may be a N-terminal-truncated variant (CPE-ΔN), which may comprise the amino acid sequence as set forth in SEQ ID NO:14.

For any of the methods described herein, the CPE-encoding polynucleotide may encode a CPE disclosed herein. The CPE-encoding polynucleotide may be derived from a mouse, which may comprise a nucleic acid sequence that is at least 95% identical to SEQ ID NO:1. One example of a mouse CPE coding sequence may be a native coding sequence, which may comprise the nucleic acid sequence as set forth in SEQ ID NO:1. Another example of a mouse CPE coding sequence may be modified from the native sequence, which may comprise the nucleic acid sequence as set forth in SEQ ID NO:5. This modified sequence encodes a mouse CPE mutant (CPE-E342Q). Still another example of a modified mouse CPE coding sequence may comprise the nucleic acid sequence as set forth in SEQ ID NO:10. This modified sequence encodes a N-terminal-truncated variant (CPE-ΔN).

Alternatively, for any of the methods described herein, the CPE-encoding polynucleotide may be derived from a human, which may comprise a nucleic acid sequence that is at least 95% identical to SEQ ID NO:3. One example of a human CPE coding sequence may be a native coding sequence, which may comprise the nucleic acid sequence as set forth in SEQ ID NO:3. Another example of a human CPE coding sequence may be modified from the native sequence, which may comprise the nucleic acid sequence as set forth in SEQ ID NO:7. This modified sequence encodes a human CPE mutant (hCPE-E342Q). Still another example of a modified mouse CPE coding sequence may comprise the nucleic acid sequence as set forth in SEQ ID NO:13. This modified sequence encodes a human N-terminal-truncated variant (CPE-ΔN).

In some embodiments, the CPE-encoding polynucleotide may be contained in an expression vector, which functions to deliver the polynucleotide to the subject. By way of non-limiting example, the expression vector may be a viral vector, which may be an adeno-associated virus (AAV) construct. The AAV construct may be an AAV1/2 hybrid construct, an AAV9 construct, or a variant of the foregoing.

Any neurodegenerative disease where there is neuronal cell death may be treatable by any of the methods described herein, which help nerve cells survive. In some embodiments, the neurodegenerative disease is Alzheimer's disease (AD), Parkinson's disease (PD), dementia, frontotemporal dementia (FTD), depression, bipolar disorder, amyotrophic lateral sclerosis (ALS), spinal cord injury, traumatic brain injury (TBI), stroke, ischemia, or Down's syndrome.

In some embodiments, the pharmaceutical composition may be administered systemically or via injection into the brain of the subject. By way of non-limiting example, the pharmaceutical composition may be administered via injection directly into the hippocampus of the subject. In some embodiments, the pharmaceutical composition may be administered via nasal spray to the subject. In some embodiments, the pharmaceutical composition may be administered via extracellular vesicles into the cerebrospinal fluid of the subject. For any of the administration routes described herein, the pharmaceutical composition may be administered at a dose that is effective to produce about 40% to about 100% increased level of CPE in the neurons of the subject.

For any of the methods described herein, it may further comprise administering a second neuroprotective factor to the subject.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing aspects and other features of the invention are explained in the following description, taken in connection with the accompanying drawings, wherein:

FIG. 1A shows the plasmid map of AAV-BASIC-EGFP construct. FIG. 1B shows the plasmid map of an AAV1/2 hybrid construct.

FIGS. 2A-2B show hippocampal CPE expressions at different ages of wild-type (WT) and 3×Tg-AD mice. FIG. 2A shows CPE expression of WT and 3×Tg-AD mice at 3, 4.5 and 6.5 months of age from Western blot analysis. FIG. 2B is a bar graph showing the direct comparison of CPE expression between WT and 3×Tg-AD mice at 3, 4.5 and 6.5 months of age. The values represent the mean±SEM (3M WT or 3×Tg-AD, n=4 mice, 4.5M WT or 3×Tg-AD, n=5 mice; 6.5 M WT or 3×Tg-AD, n=4 mice).

FIGS. 3A-3F show 3×Tg-AD mice hippocampal CPE expressions after injection of AAV-CPE constructs in comparison with control groups injected with AAV-GFP. FIGS. 3A-3B show CPE expression 1 week after the injection from Western blot analysis (FIG. 3A) or represented by a bar graph (FIG. 3B). FIGS. 3C-3D show CPE expression 8 weeks after the injection from Western blot analysis (FIG. 3C) or represented by a bar graph (FIG. 3D). FIGS. 3E-3F show CPE expression 16 week after the injection from Western blot analysis (FIG. 3E) or represented by a bar graph (FIG. 3F). The values represent the mean±SEM (1 week: GFP control n=4 mice, CPE aav n=5 mice; 8 weeks GFP control n=5 mice, CPE aav n=5 mice; 16 weeks GFP control n=4 mice, CPE aav n=5 mice). *p<0.05 compared with respective control groups. As used herein in the context of 3×Tg-AD mouse experiments, the term “WT” means that the mouse is a non-3×Tg mouse, and may be a B6129SF2/J mouse strain (JAX stock #101045).

FIGS. 4A-4C show results of the object recognition test for memory. FIG. 4A shows the recognition index in the CPE treated 3×Tg-AD mice in comparison to the 3×Tg-AD mice injected with AAV-GFP. FIG. 4B shows the recognition index in the WT mice injected with AAV-CPE in comparison to the WT mice injected with AAV-GFP. FIG. 4C shows the recognition index in the 3×Tg-AD mice injected with AAV-CPE in comparison to WT mice injected with AAV-CPE. The values represent the mean±SEM (WT-GFP=12 mice, WT-CPE=15 mice, 3TG-GFP=12 mice, 3TG-CPE=14 mice,). Student t test *p<0.05 for 3TG-GFP compared with 3TG-CPE.

FIGS. 5A-5B show the results of the Moris Water maze test. FIG. 5A shows the learning curves of 3TG-AD mice injected with AAV-CPE or AAV-GFP in comparison with WT mice injected with AAV-CPE or AAV-GFP as represented by latency over a period of several days. FIG. 5B shows the memory function of 3TG-AD mice injected with AAV-CPE or AAV-GFP in comparison with WT mice injected with AAV-CPE or AAV-GFP as represented by the time mice spent in each quadrant. One-way ANOVA analysis followed by Tukey's post-hoc multiple comparison test. For FIG. 5A, [F(3,48)=8.699, p=0.0001], *p<0.05 for 3TG-GFP compared with 3TG-CPE group; for FIG. 5B, [F(15,192)=8.03, p<0.0001],*p<0.05 for 3TG-GFP compared with 3TG-CPE group. The values represent the mean±SEM (WT-GFP=12 mice, WT-CPE=15 mice, 3TG-GFP control=11 mice, 3TG-CPE=14 mice).

FIGS. 6A-6B show effect of AAV-CPE injection on hyperphosphorylation of tau in 3TG-GFP mice. FIG. 6A shows expression of phosphorylated tau (pTau) and tau in 3×Tg-AD mice injected with AAV-GFP or AAV-CPE in comparison to WT mice injected with AAV-GFP or AAV-CPE from Western blot analysis. FIG. 6B is a bar graph showing pTau/tau in percentage of control (WT mice injected with AAV-GFP or AAV-CPE) in 3×Tg-AD mice injected with AAV-GFP or AAV-CPE. One-way ANOVA analysis followed by Tukey's post-hoc multiple comparison test. [F(3,13)=7.303, p=0.0027], *p<0.05 for 3TG-CPE compared with 3TG-GFP group. The values represent the mean±SEM, n=5 mice per group.

FIGS. 7A-7B show CPE protects human neurons against oxidative and neurotoxic stress in vitro. FIG. 7A shows neuroprotective effect of CPE in human neurons against H2O2-induced cytotoxic stress assessed by lactic dehydrogenase assay. FIG. 7B shows neuroprotective effect of CPE in human neurons against glutamate-induced neurotoxic stress assessed by lactic dehydrogenase assay. One-way ANOVA analysis followed by Tukey's post hoc multiple comparison test. For H2O2 experiments (FIG. 7A) [F (2,6)=363.2, p<0.0001]*p<0.0001 for CPE+H2O2 compared to H2O2. For glutamate experiments (FIG. 7B) [F (2,6)=117.1, p<0.0001]*p<0.0001 for CPE+glutamate compared to glutamate. Values are mean±SD, N=3.

FIG. 8A-B. Novel object recognition test after hippocampal delivery of AAV-CPEₕᵤₘₐₙ in post-symptomatic 3×Tg-AD mice. Post-symptomatic 3×Tg-AD mice received bilateral hippocampal injections of AAV-GFP or AAV-human NF-α1/CPE at age 6 months and were evaluated for memory retention by the Novel Object Recognition test at age of 11 months. Overexpression of CPE prevented the cognitive dysfunction of 3×Tg AD mice in novel object recognition test. n=11 for nonTg+GFP, n=6 for 3×Tg+GFP, n=6 for 3×Tg+CPE. t-test, *p=0.024 for 3×Tg+CPE compared with 3×Tg+GFP (FIG. 8A), ns: not significant for p=0.172 for 3×Tg+CPE compared with nonTg+GFP (FIG. 8B). Values are mean±SEM.

FIG. 9A. Representative Western blot and quantification of phosphorylated Tau expression in the hippocampus of nonTg+GFP, 3×Tg+GFP and 3×Tg+CPE mice at age of ~8 months. Phosphorylated tau was increased in the hippocampus of 3×Tg+GFP in comparison with nonTg+GFP, *p=0.0075; overexpression of CPE in the 3×Tg+CPE mice significantly reduced the phosphorylated tau, #p=0.0425. One-way ANOVA analysis followed by Tukey's post-hoc multiple comparison test, [F_{(2,12)}=7.497, p=0.0077]. n=5 mice per genotype. The values are the mean±SEM.

FIG. 9B. Representative Western blot and quantification of β-amyloid precursor (APP) expression in the hippocampus of nonTg+GFP, 3×Tg+GFP and 3×Tg+CPE mice at ~8 months of age. β-amyloid precursor was significantly increased in 3×Tg+GFP, *p=0.0039; but decreased with overexpression of CPE in 3×Tg+CPE mice, #p=0.0137. One-way ANOVA analysis followed by Tukey's post-hoc multiple comparison test, [F_{(2,12)}=9.622, p=0.0032]. n=5 mice per genotype. The values are the mean±SEM.

FIG. 9C. Representative image of β-amyloid precursor expression in the hippocampus of nonTg+GFP, 3×Tg+GFP and 3×Tg+CPE mice at age of ~8 months. Magnification 2×, scale bar=1 mm. Inset. β-amyloid precursor expression in CA1 area magnification 20×, scale bar=50 m.

FIG. 9D. β-amyloid40 in the hippocampus of nonTg+GFP, 3×Tg+GFP and 3×Tg+CPE mice at age of ~8 months. Soluble forms of β-amyloid40 were increased in 3×Tg+GFP and 3×Tg+CPE mice. *p=0.0003 for 3×Tg+GFP, and #p=0.0045 for 3×Tg+CPE in comparison with nonTg+GFP. One-way ANOVA analysis followed by Tukey's post-hoc multiple comparison test, [F_{(2,12)}=16.39, p=0.0004]. Similarly, insoluble forms of β-amyloid42 were also increased in 3×Tg+GFP and 3×Tg+CPE mice, *p=0.0013 for 3×Tg+GFP, and #p=0.0073 for 3×Tg+CPE in comparison with nonTg+GFP. One-way ANOVA analysis followed by Tukey's post-hoc multiple comparison test, [F_{(2,12)}=12.44, p=0.0012].

FIG. 9E. β-amyloid42 in the hippocampus of nonTg+GFP, 3×Tg+GFP and 3×Tg+CPE mice at age of ~8 months. The soluble form of β-amyloid42 was increased in 3×Tg+GFP, *p=0.0176 in comparison with nonTg+GFP mice. One-way ANOVA analysis followed by Tukey's post-hoc multiple comparison test, [F (2,12)=5.602, p=0.0191]. The insoluble form of β-amyloid42 was also increased in 3×Tg+GFP, *p=0.0001 in comparison with nonTg+GFP mice; however, overexpression of CPE in 3×Tg+CPE mice significantly reduced insoluble form of β-amyloid42 in comparison with 3×Tg+GFP mice #p=0.0159. One-way ANOVA analysis followed by Tukey's post-hoc multiple comparison test, [F_{(2,12)}=18.97, p=0.0002]. n=5 mice per genotype. The values are the mean±SEM.

FIG. 10A. Pre-symptomatic 3×Tg-AD mice received bilateral hippocampal injections of AAV-GFP, or AAV-human CPE or AAV-human CPE-E342Q at age 2 months and were evaluated for learning and memory retention by the Morris water maze test at age of 7 months when the 3×Tg-AD mice have developed cognitive dysfunction. Overexpression of human CPE or human CPE-E342Q prevented learning impairment of 3×Tg-AD mice in Morris water maze. 3×Tg+GFP mice displayed longer latency compared with nonTg+GFP (p=0.0212), 3×Tg+hCPE (p=0.0318) or 3×Tg+hCPE-E342Q (p=0.0178) on day5. t test. Values are mean±SEM.

FIG. 10B. 3×Tg+GFP mice spent less time in the target area NE, and more time in non-target areas. NonTg+GFP, nonTg+hE342Q, 3×Tg+hCPE and 3×Tg+hE342Q mice displayed a similar pattern of time in non-target quadrants and target quadrant. 3×Tg+hCPE and 3×Tg+hE342Q mice spent more time in target quadrant (NE) in comparison to 3×Tg+GFP. In NE target quadrant, 3×Tg+hCPE and 3×Tg+hE342Q mice spent more time in the NE target quadrant, similar to nonTg+GFP and nonTg+E342Q mice, as compared to 3×Tg+GFP mice. One way ANOVA analysis followed by Tukey's post-hoc comparison test, F_{(4,49)}=6.900 *P<0.05 for 3×Tg+GFP compared with either nonTg+GFP, 3×Tg+hCPE or 3×Tg+hE342Q in target area. n=10 for nonTg+GFP, n=12 for nonTg+hE342Q, n=10 for 3×Tg+GFP, n=11 for 3×Tg+hCPE and n=11 for 3×Tg+hE342Q. Values are mean±SEM.

### DETAILED DESCRIPTION

CPE/NF-α1 was previously shown to be acting as an extracellular trophin to protect cultured neurons (brain cells) under induced oxidative stress from dying. Mutant mice lacking CPE/NF-α1 showed severe degeneration of hippocampal neurons as well as memory and learning deficits when subjected to emotional and physical stress. Humans having mutations in the CPE/NF-α1 gene which resulted in lack of CPE/NF-α1 expression also exhibited deficits in learning and memory, and one of them developed Alzheimer's Disease (AD).

The inventors have discovered, surprisingly, that CPE/NF-α1 can be used to treat and/or prevent one or more symptoms of AD. Several mouse models that harbor mutations in human genes known to cause AD are available for use in AD research and serve as models of the typical symptoms that are associated with human AD. As described herein, the inventors used AD mice harboring three known genes that cause AD (i.e., “APP Swedish”, “MAPT P301L”, and “PSEN1 M146V”) and treated them by injecting a virus carrying the CPE/NF-α1 gene into the hippocampus before the mice showed AD symptoms at 2 months of age. The treatment effectively prevented development of AD. No deficits in memory and learning were observed in mice treated with CPE after 5 months, unlike control (untreated) AD mice, which showed severe cognitive dysfunction at the same age. The inventors also have also demonstrated that cultured human neurons survived oxidative and neurotoxic stress when treated with CPE/NF-α1.

A clinical trial using gene therapy approach to deliver brain derived neurotrophic factor (BDNF) to the brain was initiated by UCSD to treat AD patients, since it improved cognitive function in aging mice and primates. The inventors have additionally made the surprising discovery that CPE/NF-α1 effectively prevented stress-induced hippocampal cell death and cognitive dysfunction in mice, including those expressing normal levels of BNDF. These findings suggest that delivering CPE/NF-α1 to the hippocampus, including through gene delivery, thereby overexpressing CPE/NF-α1 in the hippocampus, is likely to be more effective than BDNF in preventing and/or treating AD in humans.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Treatment” or “treating,” when referring to protection of an animal from a disease, means suppressing, repressing, reducing, or completely eliminating the disease. Suppressing the disease involves administering a composition of the present invention to an animal after induction of the disease but before its clinical appearance. Repressing the disease involves administering a composition of the present invention to an animal after clinical appearance of the disease. “Preventing” the disease involves administering a composition of the present disclosure to an animal prior to onset of the disease.

### 2. Compositions

a. CPE/NF-1α

Provided herein is a CPE protein. The CPE protein may have one or more biological activities, including protecting CA3 neurons against stress-induced cell death, and cleavage of C-terminal basic amino acids from intermediates generated by proprotein convertases' action on prohormones and pro-neuropeptides. When mice are subjected to short-term stress, the hippocampus makes more CPE/NF-α1 and plays a role in preventing depression (anti-depressant) through upregulating FGF2 expression which increases neurogenesis (Murthy et al., Endocrinology, 2013, 154(9): 3284-3293; Cheng et al., Molecular Psychiatry, 2015, 20(6): 744-754).

The CPE protein or gene may be derived from an animal, such as a mammal, which may be a mouse, monkey, ape, or human. The CPE protein may be wild-type or mutant. By way of non-limiting examples, the CPE protein may be a mouse CPE protein, which may comprise an amino acid sequence that is at least 90, 91, 92, 93, 94, or 95% identical, particularly at least 95% identical, to SEQ ID NO:2. In particular, the wild-type CPE protein may comprise the amino acid sequence as set forth in SEQ ID NO:2.

The mouse CPE protein may comprise one or more mutations. In one example, the mutant CPE may be CPE-E342Q, in which the amino acid residue of Glutamate (E) at position 342 of the native (wild-type) amino acid sequence has been changed to amino acid residue Glutamine (Q). By way of non-limiting example, the mouse CPE-E342Q mutant may comprise the amino acid sequence as set forth in SEQ ID NO:6.

The mutant CPE may be an N-terminal-truncated variant of CPE/NF-la. In one example, the mutant CPE may be 40-kDa CPE/NF-la-AN, which has been identified to regulate expression of important neurodevelopmental genes (Xiao et al., 2019, Frontiers in Neuroscience 13:243, the contents of which are incorporated herein by reference). In particular, the 40-kDa CPE/NF-1α-ΔN (or 40-kDa CPE-ΔN) has been identified to have an important, enzymatically independent role in the regulation of genes critical for neurodevelopment (Xiao, et al., 2019, FASEB J., 33(1): 808-820; Qin et al., 2014, PLoS One DOI:10.1371/journal.pone.0112996; the contents of both of which are incorporated herein by reference). By way of non-limiting example, the 40 kDa CPE-ΔN may comprise the amino acid sequence as set forth in SEQ ID NO:11.

Alternatively, the CPE protein may be a human CPE protein, which may comprise an amino acid sequence that is at least 90, 91, 92, 93, 94, or 95% identical, particularly at least 95% identical, to SEQ ID NO:4. In particular, the CPE protein may be a wild-type human CPE protein comprising the amino acid sequence set forth in SEQ ID NO:4.

The human CPE may comprise one or more mutations. In one example, the mutant CPE may be hCPE-E342Q, in which the amino acid residue of glutamate (E) at position 342 of the native amino acid sequence has been changed to amino acid residue glutamine (Q). By way of non-limiting example, hCPE-E342Q may comprise the amino acid sequence set forth in SEQ ID NO:8.

The mutant CPE may be an N-terminal-truncated variant of CPE (CPE/NF-la-AN or CPE-ΔN). By way of non-limiting example, the human CPE-ΔN may comprise the amino acid sequence set forth in SEQ ID NO:14.

Amino acid variations of the CPE proteins described herein may be made based on relative similarity of amino acid side chain substituents such as hydrophobicity, hydrophilicity, charge, and size. Based on analysis of sizes, shapes, and types of amino acid side chain substituents, arginine, lysine, and histidine are all positively charged residues; alanine, glycine, and serine have similar sizes; and phenylalanine, tryptophan, and tyrosine have similar shapes. As such, arginine, lysine, and histidine; alanine, glycine, and serine; and phenylalanine, tryptophan, and tyrosine are considered as biologically functional equivalents.

In introducing variations, the hydropathic index of amino acids may be considered. Each amino acid has been assigned hydropathic index depending on its hydrophobicity and charge: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The hydropathic amino acid index is very important in conferring the interactive biological function on a protein. It is known that substitution with an amino acid having similar hydropathic index allows a protein to retain similar biological activity. In a case where variations are introduced with reference to the hydropathic index, substitutions are made between amino acids that exhibit a hydropathic index difference of preferably within ±2, more preferably within ±1, and even more preferably within ±0.5.

Meanwhile, it is also well known that substitutions between amino acids having similar hydrophilicity values result in proteins with equivalent biological activity. As disclosed in U.S. Pat. No. 4,554,101, respective amino acid residues have been assigned the following hydrophilicity values: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In a case where variations are introduced with reference to the hydrophilicity values, substitutions may be made between amino acids that exhibit a hydrophilicity value difference of preferably within ±2, more preferably within ±1, and even more preferably within ±0.5.

Amino acid exchanges in proteins which do not entirely alter activity of a molecule are known in the art (H. Neurath, R. L. Hill, The Proteins, Academic Press, New York (1979)). The most commonly occurring exchanges are exchanges between amino acid residues Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Gln/Glu.

Given the above-described variations with biologically equivalent activity, it is anticipated that CPE protein disclosed herein may also include sequences that exhibit substantial identities to the CPE sequences described herein.

As used herein, the term “substantial identity” refers to a sequence showing at least 60% homology, more preferably 70% homology, even more preferably 80% homology, and most preferably 90% homology when the sequence is aligned with any other sequence so that they maximally correspond to each other, and the aligned sequence is analyzed by using an algorithm typically used in the art. Alignment methods for comparison of sequences are known in the art. Various methods and algorithms for alignment are disclosed in Smith and Waterman, Adv. Appl. Math. 2:482 (1981); Needleman and Wunsch, J. Mol. Bio. 48:443 (1970); Pearson and Lipman, Methods in Mol. Biol. 24: 307-31 (1988); Higgins and Sharp, Gene 73:237-44 (1988); Higgins and Sharp, CABIOS 5:151-3 (1989); Corpet et al., Nuc. Acids Res. 16:10881-90 (1988); Huang et al., Comp. Appl. BioSci. 8:155-65 (1992); and Pearson et al., Meth. Mol. Biol. 24:307-31 (1994). NCBI Basic Local Alignment Search Tool (BLAST) (Altschul et al., J. Mol. Biol. 215: 403-10 (1990)) is accessible from the National Center for Biological Information (NBCI), or the like, and may be used in conjunction with sequencing programs, such as blastp, blasm, blastx, tblastn, and tblastx, on the internet.

Further provided herein is a polynucleotide encoding the CPE protein or a polynucleotide comprising the CPE coding sequence. By way of non-limiting examples, the CPE-encoding polynucleotide or the CPE coding sequence may be derived from a mouse, which may comprise a nucleic acid sequence that is at least 90, 91, 92, 93, 94, or 95% identical, particularly at least 95% identical, to SEQ ID NO:5. One example of a CPE-encoding polynucleotide or a CPE coding sequence may comprise a native mouse sequence, which may comprise the nucleic acid sequence as set forth in SEQ ID NO:5. Another example of a CPE-encoding polynucleotide or a CPE coding sequence may comprise a mutated mouse sequence. In particular, a CPE-encoding polynucleotide or a CPE coding sequence may comprise the nucleic acid sequence set forth in SEQ ID NO:7. Such sequence encodes CPE-E342Q mutant.

Alternatively, the CPE-encoding polynucleotide may be derived from a human, which may comprise an amino acid sequence that is at least 90, 91, 92, 93, 94, or 95% identical, particularly at least 95% identical, to SEQ ID NO:6. In particular, the CPE-encoding polynucleotide may comprise a native human sequence, which may comprise the amino acid sequence set forth in SEQ ID NO:6.

b. CPE Delivery Systems

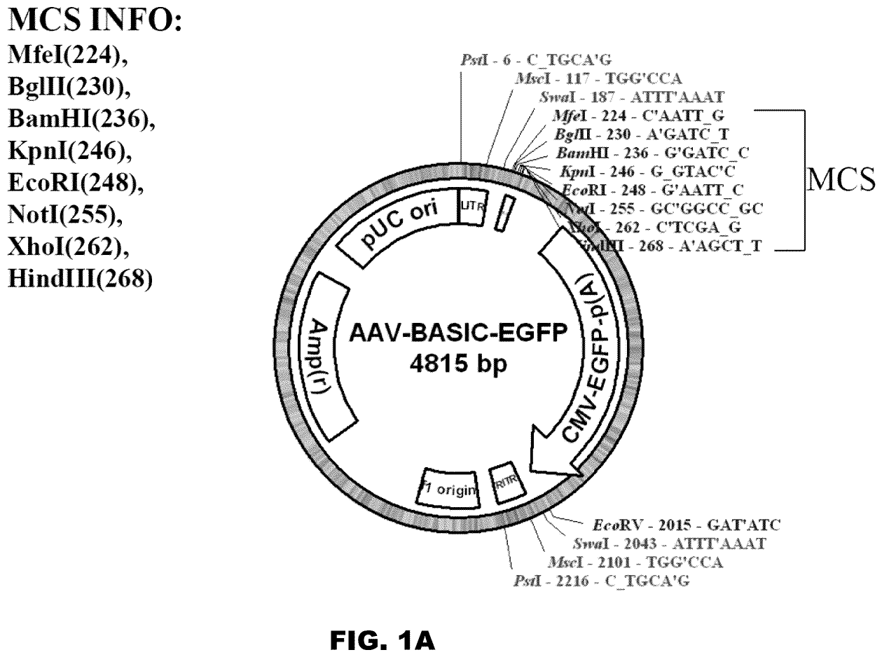

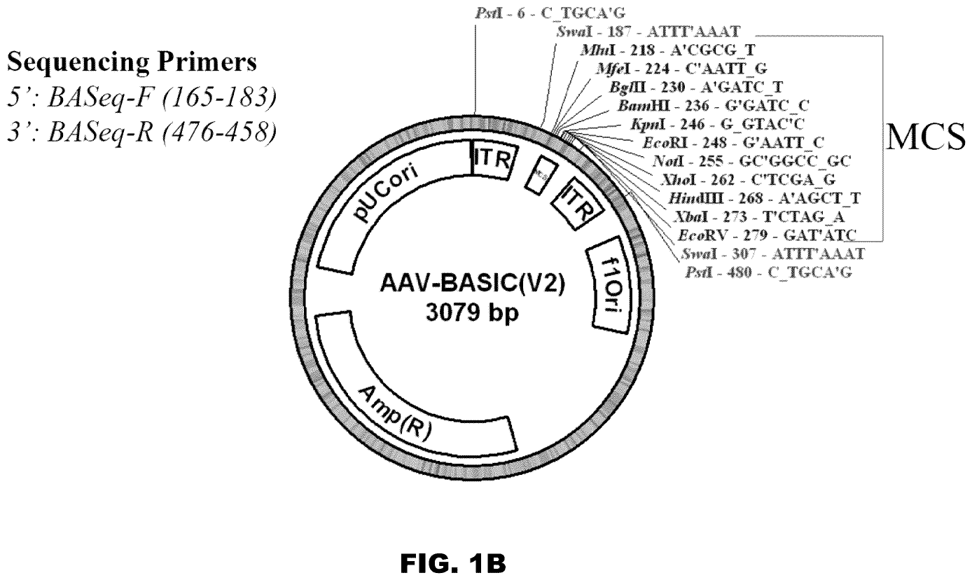

Provided herein is a delivery system for delivering the CPE protein or polynucleotide encoding the CPE protein. The delivery system may be an CPE construct that expresses a CPE protein described herein. The CPE construct may be a gene therapy system. Gene therapy systems are known in the art. The CPE construct may comprise a CPE gene or coding region thereof, which may encode the CPE protein. The CPE construct may be contained in an adeno-associated virus (AAV) system, which may be used to deliver the CPE protein. AAV systems are known to be safe for use in humans with no adverse immunoresponse. There are about 12 different serotypes for AAVs that are known in the art. For instance, AAV1 and AAV2 are two different serotypes and have different transducing efficacy to different tissue/cells. AAV2 is the most widely used one and it moderately transduces several tissue types, including the central nervous system (CNS), liver, muscle, and lung. Similarly, AAV1 can be used to transduce CNS. Within the CNS, AAV1 systems show higher transduction frequencies than AAV2 systems in all injected regions. To combine the advantages of both AAV1 and AAV2, a AAV1/2 hybrid may be used. In particular, the AAV1/2 hybrid may be generated using a transcapsidation strategy, which may involve cross-packaging inverted terminal repeats (ITRs) from one serotype into a capsid of another serotype. In one example, ITRs from AAV2 are packaged into a capsid of AAV1. By way of non-limiting example, an AAV1/2 hybrid vector may have a structure as illustrated in FIG. 1B. In one example, the CPE protein, which may be the entire mouse CPE CDS or human CPE CDS or a variant thereof described herein, may be inserted into an AAV vector to generate an AAV-CPE construct. The AAV vector may be an AAV1/2 hybrid vector, an AAV9 vector, or a variant of the foregoing.

The delivery system may also comprise a nasal spray or exosomes/extracellular vesicles. Intranasal delivery of neurotrophic factors BDNF, CNTF, EPO and NT-4 to the CNS is known to be effective for treating CNS injuries (Alcala-Barraza et al., 2010, J. Drug Target, 18(3): 179-190). Nasal sprays may be used to deliver CPE/NF-α1 protein or mRNA to the brain for treating or preventing neurodegenerative diseases. The use of custom engineered extracellular vesicles has been shown to deliver cargo such as siRNA to the brain (Cheng et al., Molecular Psychiatry, 2015, 20(6): 744-754; Xiao et al., 2021; Extracell Vesicles Circ Nucl Acids, 2: 55-79).

c. Pharmaceutical Compositions

Also provided herein is a pharmaceutical composition comprising the CPE protein, the polynucleotide encoding a CPE protein, or the CPE delivery system. The pharmaceutical composition may comprise one or more pharmaceutically acceptable carriers. In some embodiments, the pharmaceutical composition may comprise a CPE construct and a pharmaceutically acceptable carrier. In one example, the CPE construct may comprise an AAV1/2 hybrid vector as illustrated in FIG. 1B. In another, the CPE construct comprises an AAV9 vector.

As used herein, the term “pharmaceutically acceptable” refers to a molecular entity or composition that does not produce an adverse, allergic or other untoward reaction when administered to an animal or a human, as appropriate. The term “pharmaceutically acceptable carrier,” as used herein, includes any and all solvents, dispersion media, coatings, antibacterial and/or antifungal agents, isotonic and absorption delaying agents, buffers, excipients, binders, lubricants, gels, surfactants and the like, that may be used as a media for a pharmaceutically acceptable substance. In one example, the pharmaceutical composition is a liposomal formulation.

Exemplary carriers or excipients include but are not limited to, calcium carbonate, calcium phosphate, various sugars, starches, cellulose derivatives, gelatin, and polymers such as polyethylene glycols. Exemplary pharmaceutically acceptable carriers include one or more of water, saline, isotonic aqueous solutions, phosphate buffered saline, dextrose, 0.3% aqueous glycine, glycerol, ethanol and the like, as well as combinations thereof. In many cases, it will be preferable to include isotonic agents, for example, sugars, polyalcohols such as mannitol, sorbitol, or sodium chloride in the composition, or glycoproteins for enhanced stability, such as albumin, lipoprotein and globulin. Pharmaceutically acceptable carriers may further comprise minor amounts of auxiliary substances such as wetting or emulsifying agents, preservatives or buffers, which enhance the shelf life or effectiveness of the therapeutic agents.

These compositions can be sterilized by conventional sterilization techniques that are well-known to those of skill in the art. Sufficiently small liposomes, for example, can be sterilized using sterile filtration techniques.

Formulation characteristics that can be modified include, for example, the pH and the osmolality. For example, it may be desired to achieve a formulation that has a pH and osmolality similar to that of human blood or tissues to facilitate the formulation's effectiveness when administered parenterally. Alternatively, to promote the effectiveness of the disclosed compositions when administered via other administration routes, alternative characteristics may be modified.

Buffers are useful in the present invention for, among other purposes, manipulation of the total pH of the pharmaceutical formulation (especially desired for parenteral administration). A variety of buffers known in the art can be used in the present formulations, such as various salts of organic or inorganic acids, bases, or amino acids, and including various forms of citrate, phosphate, tartrate, succinate, adipate, maleate, lactate, acetate, bicarbonate, or carbonate ions. Particularly advantageous buffers for use in parenterally administered forms of the presently disclosed compositions in the present invention include sodium or potassium buffers, including sodium phosphate, potassium phosphate, sodium succinate and sodium citrate.

Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%).

In one embodiment, sodium phosphate is employed in a concentration approximating 20 mM to achieve a pH of approximately 7.0. A particularly effective sodium phosphate buffering system comprises sodium phosphate monobasic monohydrate and sodium phosphate dibasic heptahydrate. When this combination of monobasic and dibasic sodium phosphate is used, advantageous concentrations of each are about 0.5 to about 1.5 mg/ml monobasic and about 2.0 to about 4.0 mg/ml dibasic, with preferred concentrations of about 0.9 mg/ml monobasic and about 3.4 mg/ml dibasic phosphate. The pH of the formulation changes according to the amount of buffer used.

Depending upon the dosage form and intended route of administration it may alternatively be advantageous to use buffers in different concentrations or to use other additives to adjust the pH of the composition to encompass other ranges. Useful pH ranges for compositions of the present invention include a pH of about 2.0 to a pH of about 12.0.

In some embodiments, it will also be advantageous to employ surfactants in the presently disclosed formulations, where those surfactants will not be disruptive of the drug-delivery system used. Surfactants or anti-adsorbents that prove useful include polyoxyethylenesorbitans, polyoxyethylenesorbitan monolaurate, polysorbate-20, such as Tween-20™, polysorbate-80, polysorbate-20, hydroxycellulose, genapol and BRIJ surfactants. By way of example, when any surfactant is employed in the present invention to produce a parenterally administrable composition, it is advantageous to use it in a concentration of about 0.01 to about 0.5 mg/ml.

Additional useful additives are readily determined by those of skill in the art, according to particular needs or intended uses of the compositions and formulator. One such particularly useful additional substance is sodium chloride, which is useful for adjusting the osmolality of the formulations to achieve the desired resulting osmolality. Particularly preferred osmolalities for parenteral administration of the disclosed compositions are in the range of about 270 to about 330 mOsm/kg. The optimal osmolality for parenterally administered compositions, particularly injectables, is approximately 3000 sm/kg and achievable by the use of sodium chloride in concentrations of about 6.5 to about 7.5 mg/ml with a sodium chloride concentration of about 7.0 mg/ml being particularly effective.

The pharmaceutical composition may be in the form of tablets or lozenges formulated in a conventional manner. For example, tablets and capsules for oral administration may contain conventional excipients may be binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers may be lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants may be potato starch and sodium starch glycollate. Wetting agents may be sodium lauryl sulfate. Tablets may be coated according to methods well known in the art.

The pharmaceutical composition may also be liquid formulations such as aqueous or oily suspensions, solutions, emulsions, syrups, and elixirs. The pharmaceutical composition may also be formulated as a dry product for constitution with water or other suitable vehicle before use. Such liquid preparations may contain additives such as suspending agents, emulsifying agents, nonaqueous vehicles and preservatives. Suspending agents may be sorbitol syrup, methyl cellulose, glucose/sugar syrup, gelatin, hydroxyethylcellulose, carboxymethyl cellulose, aluminum stearate gel, and hydrogenated edible fats. Emulsifying agents may be lecithin, sorbitan monooleate, and acacia. Nonaqueous vehicles may be edible oils, almond oil, fractionated coconut oil, oily esters, propylene glycol, and ethyl alcohol. Preservatives may be methyl or propyl p-hydroxybenzoate and sorbic acid.

The pharmaceutical composition may also be formulated as suppositories, which may contain suppository bases such as cocoa butter or glycerides. The pharmaceutical composition may also be formulated for inhalation, which may be in a form such as a solution, suspension, or emulsion that may be administered as a dry powder or in the form of an aerosol using a propellant, such as dichlorodifluoromethane or trichlorofluoromethane. Agents provided herein may also be formulated as transdermal formulations comprising aqueous or nonaqueous vehicles such as creams, ointments, lotions, pastes, medicated plaster, patch, or membrane.

The pharmaceutical composition may also be formulated for parenteral administration such as by injection, intratumor injection or continuous infusion. Formulations for injection may be in the form of suspensions, solutions, or emulsions in oily or aqueous vehicles, and may contain formulation agents including, but not limited to, suspending, stabilizing, and dispersing agents. The pharmaceutical composition may also be provided in a powder form for reconstitution with a suitable vehicle including, but not limited to, sterile, pyrogen-free water.

The pharmaceutical composition may also be formulated as a depot preparation, which may be administered by implantation or by intramuscular injection. The pharmaceutical composition may be formulated with suitable polymeric or hydrophobic materials (as an emulsion in an acceptable oil, for example), ion exchange resins, or as sparingly soluble derivatives (as a sparingly soluble salt, for example).

### 3. Methods of Treating or Preventing Onset or Progression of Neurodegenerative Diseases

Provided herein is a method of treating or preventing onset or progression of a neurodegenerative disease in a subject. The method may comprise administering a composition described herein to the subject. The subject may be a subject in need thereof. In one example, the subject suffers from or is at risk of suffering from the neurodegenerative disease. Also provided herein is a composition described herein for treating or preventing onset or progression of a neurodegenerative disease, or use of the composition in the manufacture of a medicament for treating or preventing onset or progression of a neurodegenerative disease.

a. Combination Therapy

The pharmaceutical compositions may be used alone, or in combination with a second neuroprotective factor in any of the methods described herein. By way of non-limiting example, the neuroprotective factor may be a brain derived neurotrophic factor (BDNF).

b. Neurodegenerative Diseases

“Neurodegenerative disease,” or “neurodegenerative disorder,” as used herein, may refer to a type of disease or disorder in which cells of the central nervous system stop working or die. Neurodegenerative diseases or disorders usually get worse over time. They may be genetic or may be caused by a tumor, stroke, stress or environmental factors. By way of non-limiting example, the neurodegenerative disease or disorder may be Alzheimer's disease (AD), Parkinson's disease (PD), dementia (including frontotemporal dementia [FTD]), depression, bipolar disorder, amyotrophic lateral sclerosis (ALS), spinal cord injury, traumatic brain injury (TBI), stroke, ischemia, or Down's syndrome. In particular, the neurodegenerative disorder may be AD, FTD, or ALS. These are well-known CNS amyloidoses characterized by amyloid deposition inside and outside of cells. The amyloidogenic proteins of these diseases have distinct primary sequences, and they ordinarily function as soluble proteins. The onset of these diseases may be due to aggregation and formation of amyloid, which may have a common intermolecular tertiary structure, specifically, a cross-3-sheet structure. Even more in particular, the neurodegenerative disease or disorder may be AD. The AD treatment may prevent memory loss or tau hyperphosphorylation. The treatment may also treat, or prevent or slow progression of AD. The treatment may effective against early symptoms of AD, such as cognitive impairment, or AD pathology. In particular, the neurodegenerative disease or disorder may be Parkinson's disease (PD). The PD treatment may treat, prevent, or slow aggregation of alpha-synuclein, and may prevent neurodegeneration of dopamine neurons. In particular, the neurodegenerative disease or disorder may be dementia, including Lewy body dementia and all other subtypes of dementia.

c. Administration

The compositions described herein may be administered orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular. For veterinary use, the agent may be administered as a suitably acceptable formulation in accordance with normal veterinary practice. The veterinarian can readily determine the dosing regimen and route of administration that is most appropriate for a particular animal. The pharmaceutical composition may be administered to a human patient, cat, dog, large animal, or an avian.

In some embodiments, the composition can be formulated as a depot preparation. Such long acting formulations may be administered by implantation at an appropriate site or by parenteral injection, particularly intratumoral injection or injection at a site adjacent to the brain of the subject. In particular, the composition may be administered via injection into the brain of the subject. By way of non-limiting example, the composition may be injected directly into the hippocampus of the subject for treating Alzheimer's disease or dementia. By way of non-limiting example, the composition may be injected into Substantia Nigra where dopamine neurons degenerate for treating Parkinson's disease.

In some embodiments, the composition described herein may be administered to the subject via other non-invasive methods, including but not limited to, by nasal spray.

In some embodiments, the pharmaceutical composition may be administered by injecting extracellular vesicles loaded with a composition disclosed herein, particularly an AAV-CPE construct, a CPE-encoding mRNA, or a CPE protein (native or recombinant). The extracellular vesicles may be administered into the cerebrospinal fluid or systemically. The composition may be injected intraventricularly into the cerebrospinal fluid of the subject. In one example, the delivered CPE protein is naked protein. In another example, the AAV-CPE construct, CPE-encoding mRNA, or CPE protein is incorporated into extracellular vesicles. In some other particular embodiments, the composition may be injected intraperitoneally into the subject via extracellular vesicles.

Liposomal preparations or other microemulsion delivery vehicles can be lyophilized and stored as sterile powders, preferably under vacuum, and then reconstituted in bacteriostatic water (containing, for example, benzyl alcohol preservative) or in sterile water prior to injection. Pharmaceutical compositions may be formulated for parenteral administration by injection, e.g., by bolus injection or continuous infusion.

The composition may be administered to the patient at one time or over a series of treatments and may be administered to the patient at any time from diagnosis onwards. The delivery vehicle may be administered as the sole treatment or in conjunction with other drugs or therapies useful in treating the condition in question. In some embodiments, compositions described herein may be administered in conjunction with a second neuroprotective factor. By way of non-limiting example, the neuroprotective factor may be a brain derived neurotrophic factor (BDNF).

The composition may be administered simultaneously or metronomically with other treatments. The term “simultaneous” or “simultaneously” as used herein, means that the pharmaceutical composition and other treatment be administered within 48 hours, preferably 24 hours, more preferably 12 hours, yet more preferably 6 hours, and most preferably 3 hours or less, of each other. The term “metronomically” as used herein means the administration of the agent at times different from the other treatment and at a certain frequency relative to repeat administration.

The pharmaceutical composition may be administered at any point prior to another treatment including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins, 10 mins, 9 mins, 8 mins, 7 mins., 6 mins., 5 mins., 4 mins., 3 mins, 2 mins, and 1 mins. The pharmaceutical composition may be administered at any point prior to a second treatment of the pharmaceutical composition including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

The pharmaceutical composition may be administered at any point after another treatment including about 1 min, 2 mins., 3 mins., 4 mins., 5 mins., 6 mins., 7 mins., 8 mins., 9 mins., 10 mins., 15 mins., 20 mins., 25 mins., 30 mins., 35 mins., 40 mins., 45 mins., 50 mins., 55 mins., 1 hr, 2 hr, 3 hr, 4 hr, 6 hr, 8 hr, 10 hr, 12 hr, 14 hr, 16 hr, 18 hr, 20 hr, 22 hr, 24 hr, 26 hr, 28 hr, 30 hr, 32 hr, 34 hr, 36 hr, 38 hr, 40 hr, 42 hr, 44 hr, 46 hr, 48 hr, 50 hr, 52 hr, 54 hr, 56 hr, 58 hr, 60 hr, 62 hr, 64 hr, 66 hr, 68 hr, 70 hr, 72 hr, 74 hr, 76 hr, 78 hr, 80 hr, 82 hr, 84 hr, 86 hr, 88 hr, 90 hr, 92 hr, 94 hr, 96 hr, 98 hr, 100 hr, 102 hr, 104 hr, 106 hr, 108 hr, 110 hr, 112 hr, 114 hr, 116 hr, 118 hr, and 120 hr. The pharmaceutical composition may be administered at any point prior after a pharmaceutical composition treatment of the agent including about 120 hr, 118 hr, 116 hr, 114 hr, 112 hr, 110 hr, 108 hr, 106 hr, 104 hr, 102 hr, 100 hr, 98 hr, 96 hr, 94 hr, 92 hr, 90 hr, 88 hr, 86 hr, 84 hr, 82 hr, 80 hr, 78 hr, 76 hr, 74 hr, 72 hr, 70 hr, 68 hr, 66 hr, 64 hr, 62 hr, 60 hr, 58 hr, 56 hr, 54 hr, 52 hr, 50 hr, 48 hr, 46 hr, 44 hr, 42 hr, 40 hr, 38 hr, 36 hr, 34 hr, 32 hr, 30 hr, 28 hr, 26 hr, 24 hr, 22 hr, 20 hr, 18 hr, 16 hr, 14 hr, 12 hr, 10 hr, 8 hr, 6 hr, 4 hr, 3 hr, 2 hr, 1 hr, 55 mins., 50 mins., 45 mins., 40 mins., 35 mins., 30 mins., 25 mins., 20 mins., 15 mins., 10 mins., 9 mins., 8 mins., 7 mins., 6 mins., 5 mins., 4 mins., 3 mins, 2 mins, and 1 mins.

d. Dosage

The composition described herein may be administered to a subject in need thereof in a therapeutically effective amount. The amount may be such that the level of CPE in the neurons of the subject is increased about 40% to about 100%, as compared to an untreated subject or the mean amount in a population of untreated subjects. The comparison may be against the subject before being treated with a CPE composition described herein. The therapeutically effective amount required for use in therapy varies with the nature of the condition being treated, the age/condition of the patient, etc. among other factors.

The dosages can be tested in a suitable animal model as further described below. As a general proposition, a therapeutically effective amount of CPE or other neuroprotective factor will be administered in a range from about 10 ng/kg body weight/day to about 100 mg/kg body weight/day whether by one or more administrations. In some embodiments, each therapeutic agent is administered in the range of from about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, about 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 μg/kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day. The dose regimen may achieve optimal therapeutic effect, which may occur without significant adverse effects.

In other embodiments, the composition described herein may be administered in the range of about 10 ng to about 100 ng per individual administration, about 10 ng to about 1 g per individual administration, about 10 ng to about 10 g per individual administration, about 10 ng to about 100 g per individual administration, about 10 ng to about 1 mg per individual administration, about 10 ng to about 10 mg per individual administration, about 10 ng to about 100 mg per individual administration, about 10 ng to about 1000 mg per injection, about 10 ng to about 10,000 mg per individual administration, about 100 ng to about 1 g per individual administration, about 100 ng to about 10 g per individual administration, about 100 ng to about 100 g per individual administration, about 100 ng to about 1 mg per individual administration, about 100 ng to about 10 mg per individual administration, about 100 ng to about 100 mg per individual administration, about 100 ng to about 1000 mg per injection, about 100 ng to about 10,000 mg per individual administration, about 1 g to about 10 g per individual administration, about 1 g to about 100 g per individual administration, about 1 g to about 1 mg per individual administration, about 1 g to about 10 mg per individual administration, about 1 g to about 100 mg per individual administration, about 1 g to about 1000 mg per injection, about 1 g to about 10,000 mg per individual administration, about 10 g to about 100 g per individual administration, about 10 g to about 1 mg per individual administration, about 10 g to about 10 mg per individual administration, about 10 g to about 100 mg per individual administration, about 10 g to about 1000 mg per injection, about 10 g to about 10,000 mg per individual administration, about 100 g to about 1 mg per individual administration, about 100 g to about 10 mg per individual administration, about 100 g to about 100 mg per individual administration, about 100 g to about 1000 mg per injection, about 100 g to about 10,000 mg per individual administration, about 1 mg to about 10 mg per individual administration, about 1 mg to about 100 mg per individual administration, about 1 mg to about 1000 mg per injection, about 1 mg to about 10,000 mg per individual administration, about 10 mg to about 100 mg per individual administration, about 10 mg to about 1000 mg per injection, about 10 mg to about 10,000 mg per individual administration, about 100 mg to about 1000 mg per injection, about 100 mg to about 10,000 mg per individual administration and about 1000 mg to about 10,000 mg per individual administration. The pharmaceutical composition may be administered daily, every 2, 3, 4, 5, 6 or 7 days, or every 1, 2, 3 or 4 weeks.

In other particular embodiments, the composition described herein may be administered at a dose of about 0.0006 mg/day, 0.001 mg/day, 0.003 mg/day, 0.006 mg/day, 0.01 mg/day, 0.03 mg/day, 0.06 mg/day, 0.1 mg/day, 0.3 mg/day, 0.6 mg/day, 1 mg/day, 3 mg/day, 6 mg/day, 10 mg/day, 30 mg/day, 60 mg/day, 100 mg/day, 300 mg/day, 600 mg/day, 1000 mg/day, 2000 mg/day, 5000 mg/day or 10,000 mg/day. As expected, the dosage will be dependent on the condition, size, and age of the patient.

In some embodiments, the composition described herein may be delivered by virus at a titer of 20-40 MOI. In some particular embodiments, the volume delivered may be limited to no more than 1 μl bilaterally in the hippocampus of the subject in need of such treatment.

The therapeutic agents in the compositions described herein may be formulated in a “therapeutically effective amount.” A “therapeutically effective amount” refers to an amount effective, at dosages and for periods of time necessary, to achieve the desired therapeutic result. A therapeutically effective amount of the liposomal formulation or other microemulsion drug-delivery vehicle may vary depending on the condition to be treated, the severity and course of the condition, the mode of administration, the bioavailability of the particular agent(s), the ability of the delivery vehicle to elicit a desired response in the individual, previous therapy, the age, weight and sex of the patient, the patient's clinical history and response to the antibody, the type of the fusion protein or expression vector used, discretion of the attending physician, etc. A therapeutically effective amount is also one in which any toxic or detrimental effects of the delivery vehicle is outweighed by the therapeutically beneficial effects.

The present invention has multiple aspects, illustrated by the following non-limiting examples.

### Example 1

### CPE/NF-α1 Prevents Onset of Cognitive Dysfunction and AD Pathology in 3×Tg-AD Mice

This example demonstrates that overexpression of CPE/NF-α1, including the use of a pharmaceutical composition comprising a CPE/NF-α1 construct, is an effective therapeutical drug for Alzheimer's Disease (AD).

Studies in mouse models have shown that CPE-NF-α1 plays important roles in protecting neurons from dying during severe stress and in anti-depression. During stress, glucocorticoids are secreted from the adrenals into the circulation. Glucocorticoids then circulate to various areas of the brain including the hippocampus where it stimulates glutaminergic neurons to secrete glutamate onto CPE/NF-α1 rich neurons in the CA3 region. These neurons then secrete CPE/NF-al to protect them from glutamate-induced cell death in an autocrine/paracrine manner. Since CA3 neurons are very important in cognitive function, absence of CPE/NF-α1 in CA3 neurons resulted in their degeneration and cognitive dysfunction in CPE-KO mice after stress, despite having normal levels of brain derived neurotrophic factor (BDNF), a trophin known to have neuroprotective activity, and other growth factors in the brain. The findings of the present disclosure indicate that CPE,NF-α1 is critical for protecting neurons from stress-induced cell death. Indeed, a human with a CPE mutation showed cognitive dysfunction and AD symptoms.

The neuroprotective effect was shown to be independent of CPE-enzymatic activity as evidenced by the observation that a CPE-E342Q knock-in mutant mouse that lacks CPE enzymatic activity showed neuroprotection of CA3 neurons against severe stress (Xiao et al., 2021, Translational Psychiatry, 11: 24-36). The mechanism for the neuroprotective activity of CPE/NF-α1 was elucidated from in vitro studies. CPE/NF-α1 was shown to interact with HTR1E, a serotonin receptor in human neurons which activated ERK-signaling and increased levels of BCL2, a pro-survival mitochondrial protein that protected the human neurons from oxidative-stress induced cell death (Sharma et al., 2020, The FASEB J., 34(S1): 1).

In addition, CPE-KO mice exhibited depressive-like behavior. When these mice were treated with FGF2, the depressive-like behavior was reversed. It was found that CPE/NF-α1 up-regulated FGF2 expression in the hippocampus, which then increased neurogenesis in the dentate gyrus, a mechanism known to alleviate depressive-like behavior (Cheng et al. 2015). Given the strong neuroprotective effects of CPE/NF-α1 against stress-induced degeneration of neurons, CPE/NF-α1 was used to treat AD in the present disclosure. CPE/NF-α1 was overexpressed in the hippocampus of 3×Tg-AD mouse model by injection with adeno-associated virus (AAV) carrying the CPE mRNA, at 2 months of age, before the onset of any cognitive dysfunction symptoms. The mice were tested for learning and memory behavior using the Morris Water Maze test and the Novel Object Recognition test at age ~8 months. The results showed that overexpression of CPE/NF-α1 in the hippocampus of 3×Tg-AD mice prevented the onset of cognitive impairment in memory, and tau hyperphosphorylation that causes neurofibrillary tangles, present in the untreated 3×Tg-AD mice at age 8 months.

### Materials and Methods

Animals: Male 3×Tg-AD mice (Cat #004807) and control mice (Cat #101045) were purchased from Jackson Laboratory (Bar Harbor, ME 04609). AD (3×Tg-AD) mice possessing three human transgenes associated with familial AD: PS1M146V, APPswe, and tauP301L were generated and provided by Frank LaFerla (UC Irvine, Oddo et al. 2003) to the Jackson Laboratory from where the animals were obtained. All animals were housed at NIH animal facility with free access to food and water ad libitum and controlled humidity (45%) and temperature (22° C.) under a 12 h light/dark cycle.

Stereotaxic injections of AAV-CPE or AAV-GFP in hippocampus of mice: Mice were anaesthetized, immobilized in stereotaxic apparatus and injected with adeno-associated virus (AAV)-carrying various constructs, according to the protocol approved by the Animal Care and Use Committee of NICHD, NIH. AAV viruses expressing mouse CPE or GFP (an example of AAV-GFP construct is illustrated in FIG. 1A) were bilaterally injected into the hippocampus (total 3×10⁹ viral particles, 1 μl on each side of hippocampus) according to the coordinates AP: −1.94 mm, L: ±1.0 mm, V: −1.3 mm. To validate CPE expression after injection, mice were injected at age of ~2.5 month, and sacrificed after 1 week, 8 weeks and 16 weeks for Western blot. Another group of mice were injected at age 2 months, and behavioral studies were performed at the age of 7-8 months.

Western blot: Mouse brain tissues were prepared in RIPA (Thermo-fisher, Waltham, MA) supplemented with protease and phosphatase inhibitor cocktail (Thermo-fisher, Waltham, MA) and centrifuged. Lysates were used for Western blot and run on SDS-PAGE gels and transferred onto nitrocellulose membrane. The membrane was incubated with the following primary antibodies: 1:2000 CPE (BD Biosciences San Jose, CA); 1:3000 β-actin (Cell signal, Danvers, MA); 1:3000 GFP (Abcam, Cambridge, United Kingdom); 1:2000 tau (Santa Cruz, Dallas, TX) and 1:1000 ptau (Santa Cruz, Dallas, TX) overnight, after blocking with 5% nonfat milk for 1 h, and then with secondary fluorescent conjugated anti-mouse or rabbit antibodies. The bands were visualized and quantified by the Odyssey software, version 2.1. The protein expression level for each sample was normalized to β-actin.

The novel object recognition (NOR) Test: The NOR test is a behavioral test conducted on the mice. It consists of three phases: on Day 1, mice were habituated to the experimental arena in absence of objects for 10 min; on Day 2, mice were trained twice by being placed in the experimental arena with 2 objects and were allowed to explore for 10 minutes; and on Day 3, long-term memory was tested 24 hours after training. Mice were allowed to explore the experimental arena for 10 minutes in the presence of 1 familiar and 1 novel object. The novel objects were counterbalanced in all experiments and the objects and apparatus were cleaned with 70% ethanol between trials to avoid olfactory cues. Mice were tracked by ANY-maze software (ANY-maze, Wood Dale, IL). Recognition index defined as [time exploring new object/(time exploring new object+time exploring familiar objects)]×100 were calculated.

Morris Water Maze Test: Morris water maze test is another behavioral test conducted on the mice. Such test was used to study spatial learning and memory. The test consists of a 5-day hidden platform training and 1-day probe test. Test was performed in a circular pool (diameter of 1 m) filled with water and nontoxic white paint. Video tracking and navigational parameters were analyzed with Any Maze software (ANY-maze, Wood Dale, IL). On Day 1, the mouse was placed in the pool facing towards the wall. There were four trials each day and mice were placed in a new quadrant on each trial. The hidden platform was put in the same position for all four trials. Mice would search for the platform for 1 min and were then placed on the platform for 30 seconds before being removed. If mice did not find the hidden platform, they were guided to the platform and allowed to sit on it for 30 seconds. Escape latency, the time for mice to find the hidden platform was recorded for five days. Twenty-four hours after the last training session, the hidden platform was removed, and the mice were allowed to explore the pool for 1 min. The time mice spent in each quadrant was recorded and analyzed by ANY-maze software (ANY-maze, Wood Dale, IL).

### Example 2

### Overexpression of CPE/NF-α1 in Hippocampus of 3×Tg-AD Mice by Injection of AAV-CPE

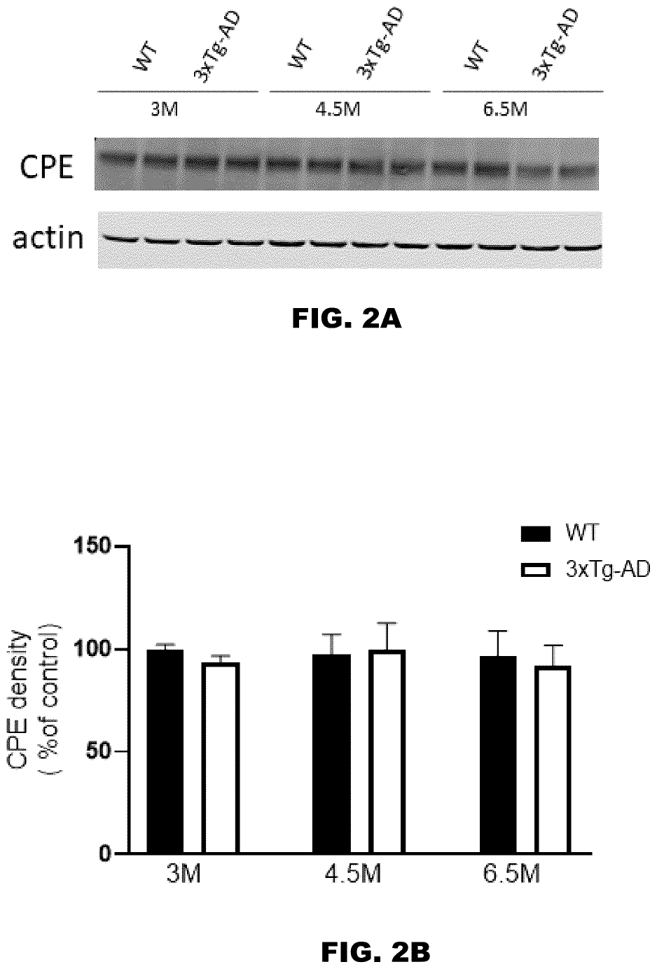

First, it was determined if 3×Tg-AD mice expressed levels of CPE/NF-α1 protein in the hippocampus comparable to that of normal wild type (WT) mice with same genetic background. As shown in FIGS. 2A-2B, there were no significant differences in CPE expression between WT and 3×Tg-AD at the age of 3 months, 4.5 months and 6.5 months. That is, at 3, 4.5 and 6.5 months of age, the level of CPE/NF-α1 protein in the hippocampus of 3×Tg-AD mice was similar to the level of CPE/NF-α1 protein in the WT mice.

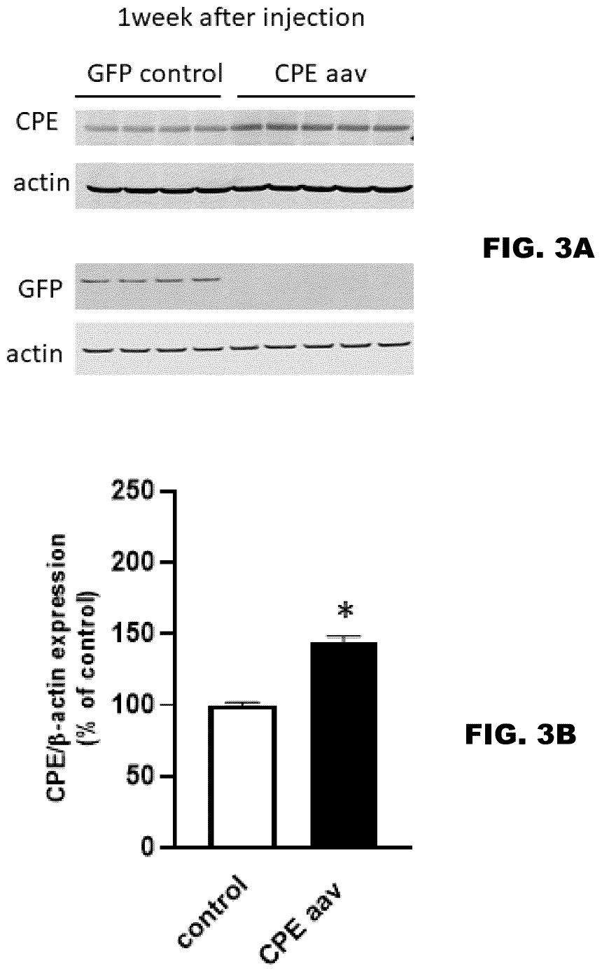

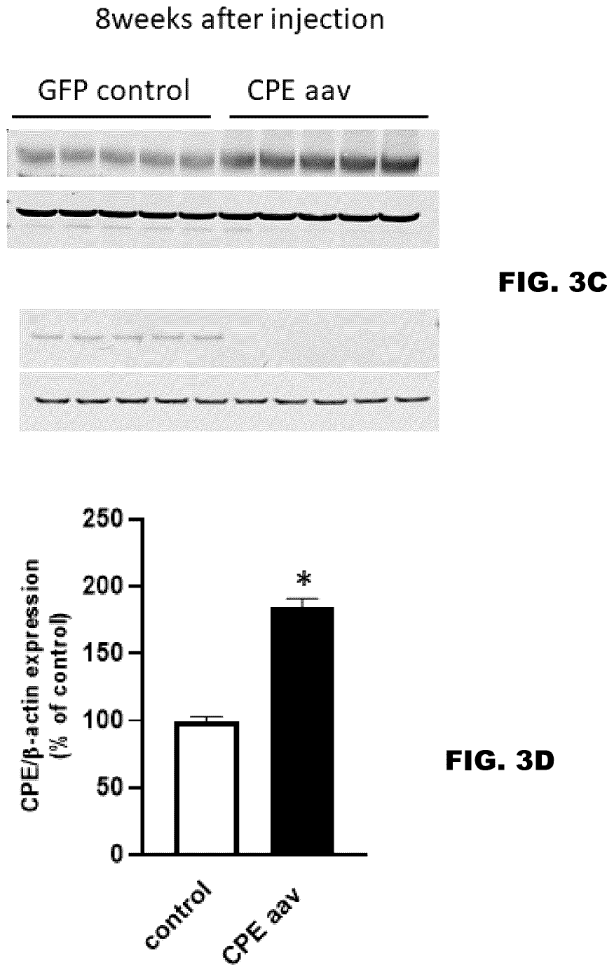

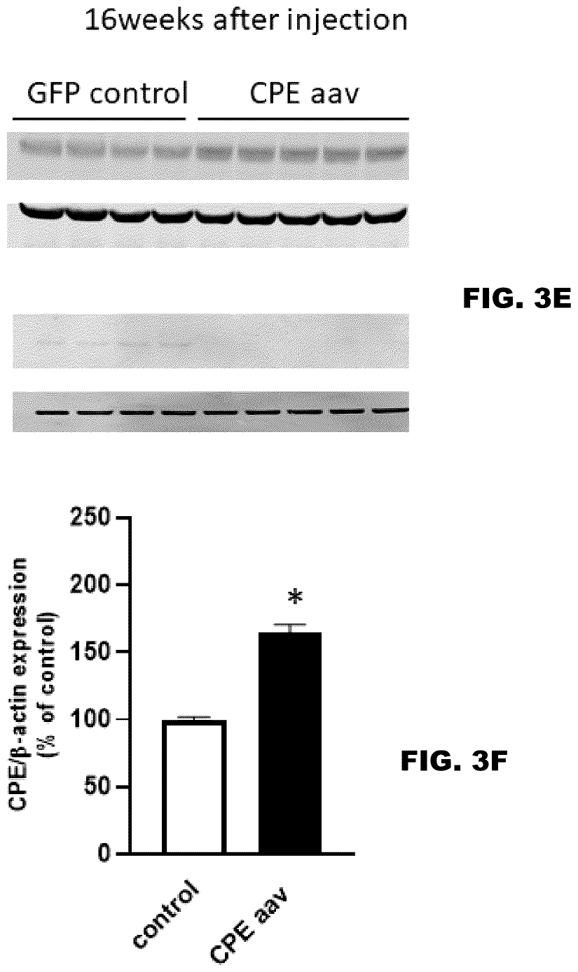

Next, the level of CPE in the hippocampus of mice at different times after injection of the AAV constructs was determined. The results show that the level of CPE protein in 3×Tg-AD mice injected with AAV-CPE significantly increased by 44.2%, 84.6%, 64.6% as compared to the control (i.e., mice injected with AAV-GFP) after 1, 8 and 16 weeks (see, FIGS. 3A-3B, FIGS. 3C-3D, and FIGS. 3E-3F, respectively). That is, stereotaxic injection of AAV-CPE enhanced hippocampal CPE expression after 1 week, 8 weeks and 16 weeks in 3×Tg-AD mice, in comparison with control groups. This suggests that elevated CPE expression levels can be maintained for at least 16 weeks after injection.

### Example 3

### Expression of CPE/NF-α1 in Hippocampus Prevents Cognitive Dysfunction in 3×Tg-AD Mice

Control mice and 3×Tg-AD mice were injected with AAV-CPE or AAV-GFP in the hippocampus at about 2 months of age. At about 7-8 months of age, mice were subjected to the novel object recognition test which is an efficient test of memory.

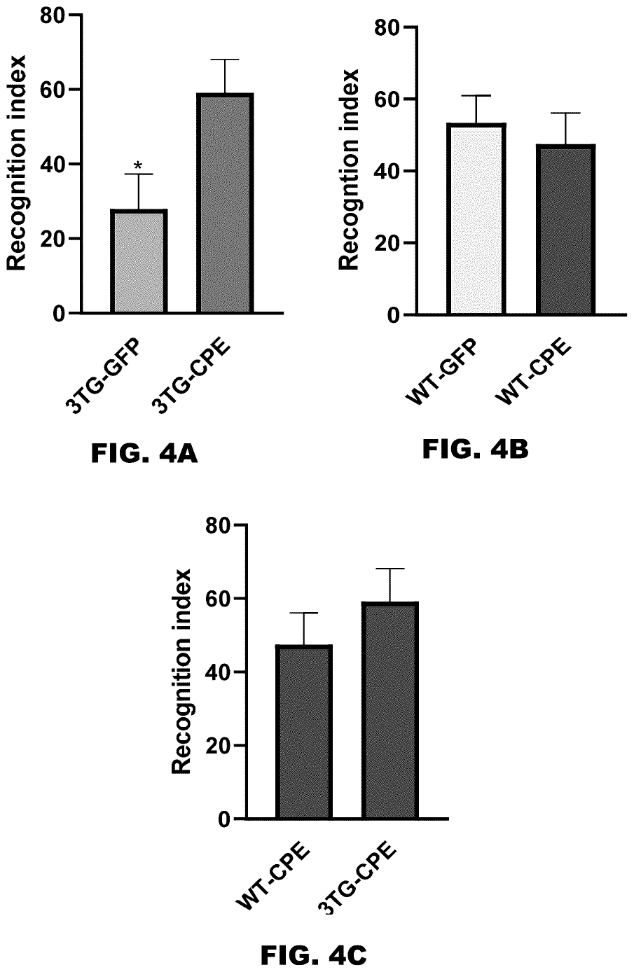

FIG. 4A shows that the recognition index in the CPE treated 3×Tg-AD mice was significantly higher compared to the mice injected with AAV-GFP, indicating poorer memory function in the non-CPE treated 3×Tg-AD mice. WT mice treated with AAV-CPE or AAV-GFP showed no significant difference in recognition index (FIG. 4B). In addition, the recognition index of the 3×Tg-AD mice treated with AAV-CPE was similar to the recognition index of the WT mice treated with AAV-CPE (FIG. 4C). These results suggest that increased CPE/NF-α1 expression in the hippocampus prevented long term memory impairment observed in the 3×Tg-AD mice at 8 months of age.

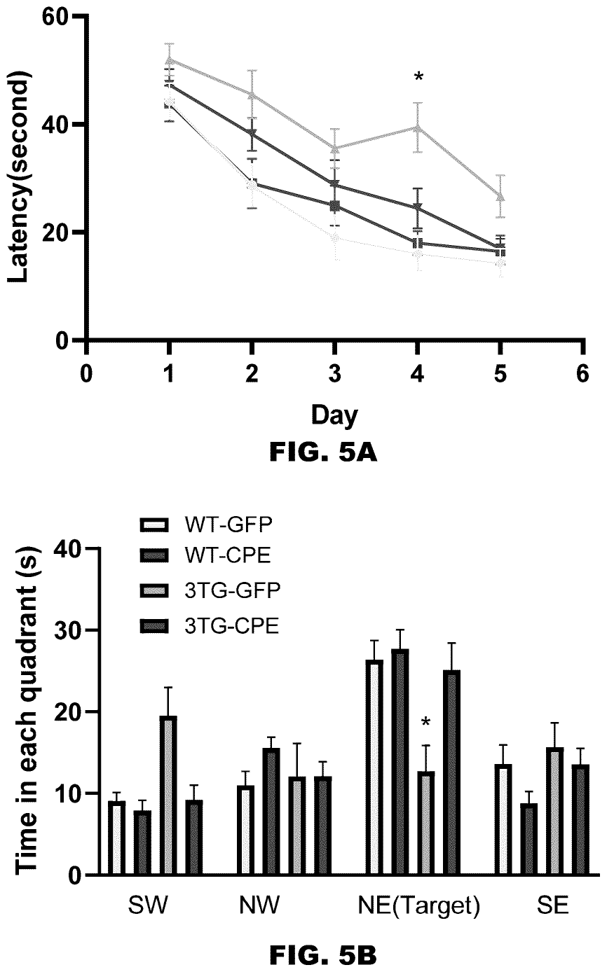

A second behavioral test for learning and memory function was performed using the Morris Water maze test to determine the effect of CPE/NF-α1 overexpression in 3×Tg-AD mice. As shown in FIG. 5A, after stereotaxic injection of AAV-CPE in the hippocampus at the age of ~2 months, 3×Tg-AD mice had a learning curve similar to the WT mice injected with AAV-CPE or AAV-GFP when tested at ~8 months of age. In contrast, 3×Tg-AD mice injected with AAV-GFP showed an abnormal learning curve, indicative of leaning deficit in these mice.

In the memory probe test, the 3×Tg-AD mice injected with AAV-GFP showed poor memory function compared to 3×Tg-AD mice injected with AAV-CPE, which behaved in a similar manner to control (WT) mice injected with AAV-GFP or AAV-CPE (see, NE (Target) in FIG. 5B). The results suggest that overexpression of CPE/NF-α1 in the hippocampus of 3×Tg-AD mice prevented the deficits in learning and memory functions observed at ~8 months of age of these mice.

### Example 4

### Expression of CPE/NF-α1 in Hippocampus Prevents Tau Phosphorylation in 3×Tg-AD Mice

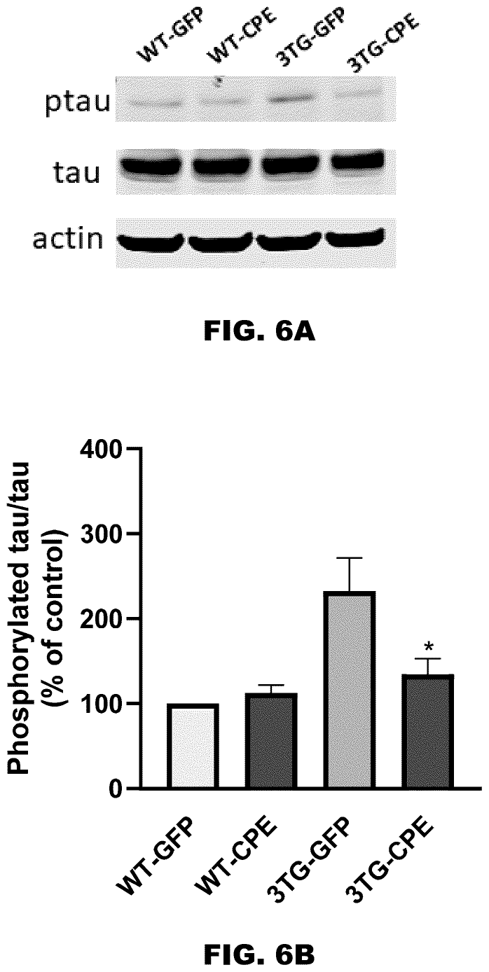

Phosphorylated tau (pTau) localized in neurofibrillary tangles in the brain of AD patients is a hallmark of the disease. As shown in FIGS. 6A-6B, 3×Tg-AD mice injected with AAV-GFP had significantly higher levels of pTau compared to 3×Tg-AD mice injected with AAV-CPE, which had levels of pTau similar to control (WT) mice injected with AAV-GFP or AAV-CPE. The results suggest that overexpression of CPE/NF-α1 in the hippocampus prevented the hyperphosphorylation of tau in the hippocampus of 3×Tg-AD mice.

### Example 5

### CPE/NF-α1 Protects Human Neurons from Cytotoxic and Neurotoxic Stress

Human primary neurons were seeded in 96 well, poly-D-lysine coated plates at a density of 13000 neurons/well in neuronal medium (Cat. #1521, ScienCell Research Laboratories, Carlsbad, CA) and cultured until they were attached to the plate. The neurons were treated overnight with 50 nM recombinant mouse CPE (custom synthesized and highly purified by GenScript, NJ, USA, Xiao et al., 2021, Translational Psychiatry, 11: 24-36). The medium was replaced with neuronal medium without growth factors and the neurons challenged with 100 μM H₂O₂ for 6 h or 40 μM glutamate (Sigma-Aldrich, St. Louis, MO) for 24 hr. Cytotoxicity was measured by the amount of LDH released using a CytoTox 96 assay kit (Promega, USA).

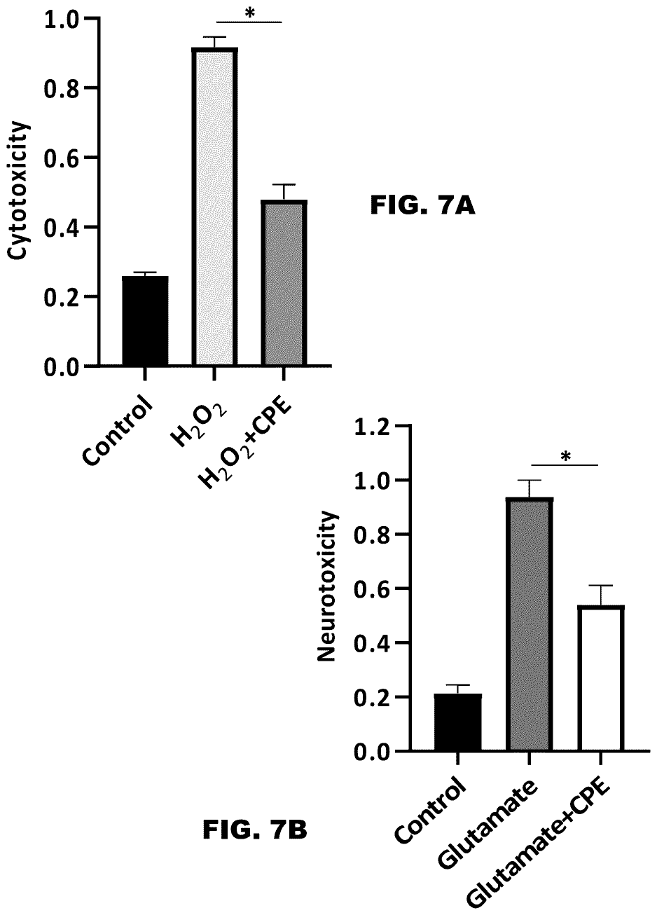

The results show that CPE/NF-α1 exhibited neuroprotective effect in human neurons against H₂O₂— induced cytotoxic (FIG. 7A) and glutamate-induced neurotoxic stress (FIG. 7B) assessed by lactic dehydrogenase assay. That is, CPE protects human neurons against oxidative and neurotoxic stress in vitro.

### DISCUSSION

The inventors showed that injection of AAV-CPEₘₒᵤₛₑ into the hippocampus of 3×Tg-AD mice at the age of ~2 months (prior to onset of AD symptoms), which led to increased CPE/NF-al expression in the 3×Tg-AD mice, effectively prevented memory loss and tau hyperphosphorylation typically found in these animals at 7-8 month of age when tested. That is, AAV-mediated delivery of mouse CPE gene into the hippocampus of 3×tg-AD mice pre-symptomatically successfully prevented these mice from developing cognitive dysfunction.

In an upcoming study, AAV carrying human CPE mRNA is to be injected in the hippocampus of 3×Tg-AD mice that have developed early symptoms of cognitive impairment and AD pathology to determine if AAV-CPE can reverse or halt the progression of the disease. The human CPE protein amino acid sequence is 96.6% identical and 97.9% similar to the mouse sequence, as such no functional difference is expected between these two molecular species. These studies will pave the way to use AAV-CPE/NF-α1 as a gene therapy approach to treat AD patients in that it inhibits the onset or progression of cognitive dysfunction and various brain pathology associated with AD.

The inventors have recently identified the human receptor for CPE as HTR1E, and demonstrated that the interaction between CPE and HTR1E protected cultured human primary neurons against oxidative stress (Sharma et al., Cell and Mol. Life Sciences 2021, 79:24). The interaction domain of CPE with HTR1E has been identified through molecular modeling studies. Future plans include identifying small molecules and CPE peptide fragments that can interact with HTR1E and act as agonists. Moreover, we have shown that CPE have effects on mitochondria to increase BCL2, a pro-survival protein, and energy (ATP) production.

The results presented herein suggest that non-invasive procedures, such as the use of custom engineered exosomes that have been shown to deliver cargo such as siRNA to the brain (Xiao et al., 2021; Extracell Vesicles Circ Nucl Acids, 2: 55-79), and nasal sprays, may be used to deliver CPE/NF-α1 protein or mRNA to the brain. The results also suggest that, CPE/NF-α1 may also be useful to treat other neurodegenerative diseases such as Parkinson's disease.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

### Example 6

### Human CPE Provides Post-Symptomatic Cognitive Improvements

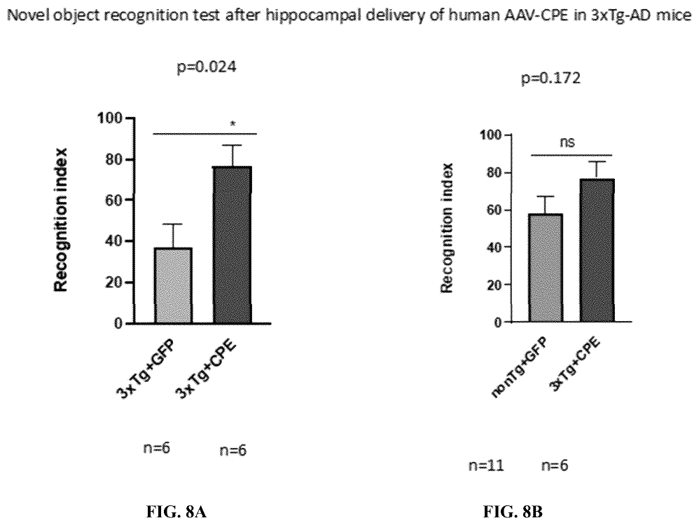

This example demonstrates that CPE can provide cognitive improvements in post-symptomatic AD. In particular, 6 month-old post-symtomatic 3×Tg-AD mice showed improvement in cognitive function after delivery of human AAV-NF-α1/CPE gene into the hippocampus. Six-month-old 3×Tg and non-Tg (control) mice were bilaterally injected with human AAV-NF-α1/CPE or AAV-GFP into the hippocampus and tested at 10-11 months of age. Using the novel object recognition (NOR) test, it was found that 3×Tg-AD mice injected with AAV-CPE (3×Tg+CPE) had a significantly higher recognition index than mice injected with AAV-GFP (3×Tg+GFP) (FIG. 8A). The 3×Tg+CPE mice had a similar recognition index to non Tg+GFP mice (FIG. 8B). This result indicates that human AAV-NF-α1/CPE treatment was able to rescue cognitive dysfunction in older postsymptomatic 3×Tg-AD mice.

### Example 7

### AAV-NF-α1/CPE Down-Regulates Hippocampal Tau Phosphorylation and APP/Aβ42 Levels in 3×Tg-AD mice

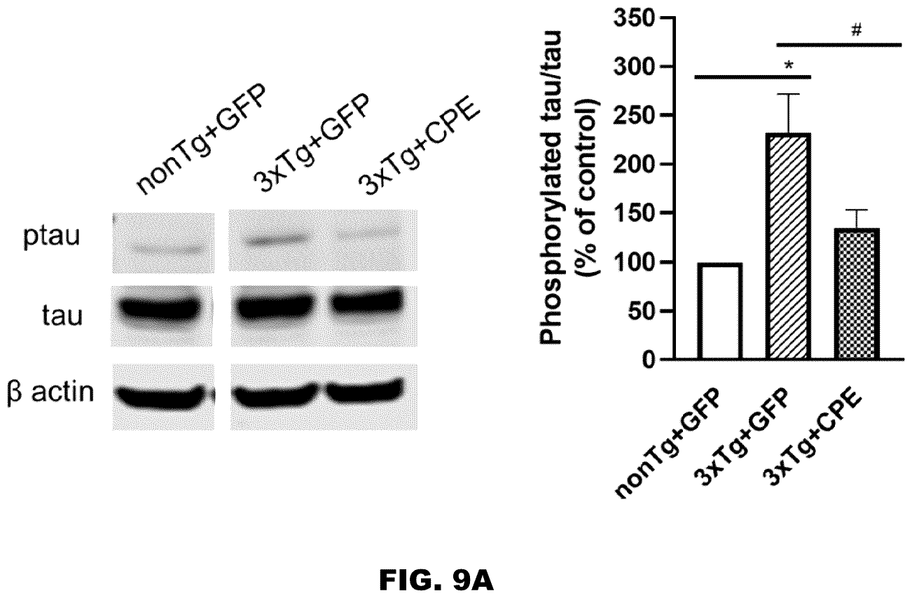

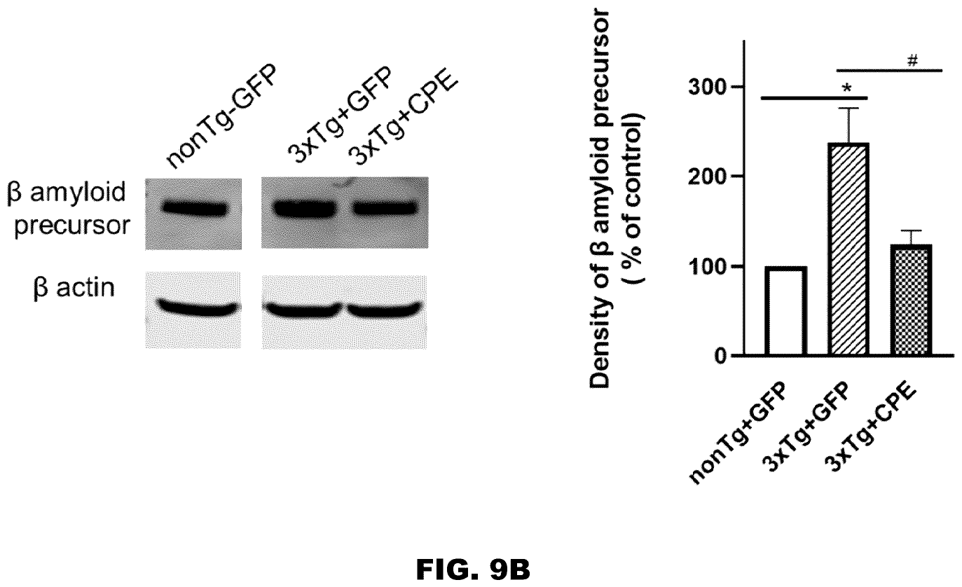

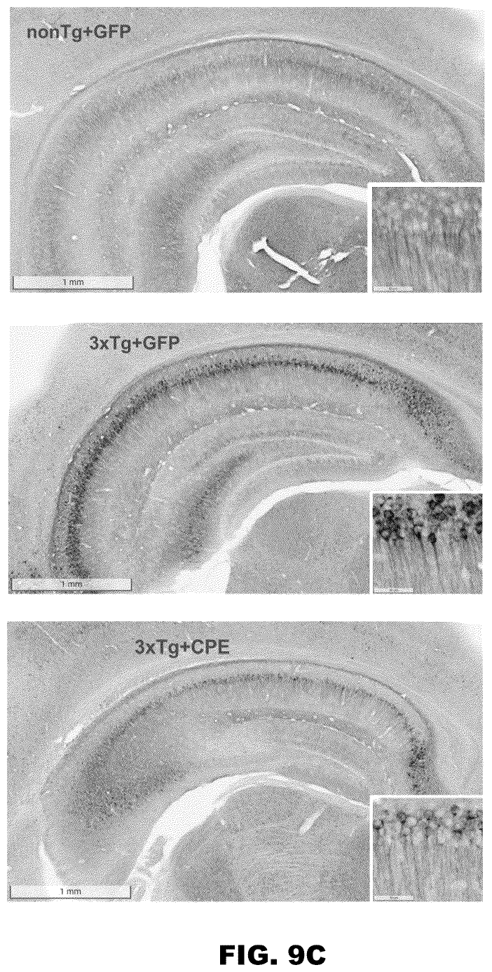

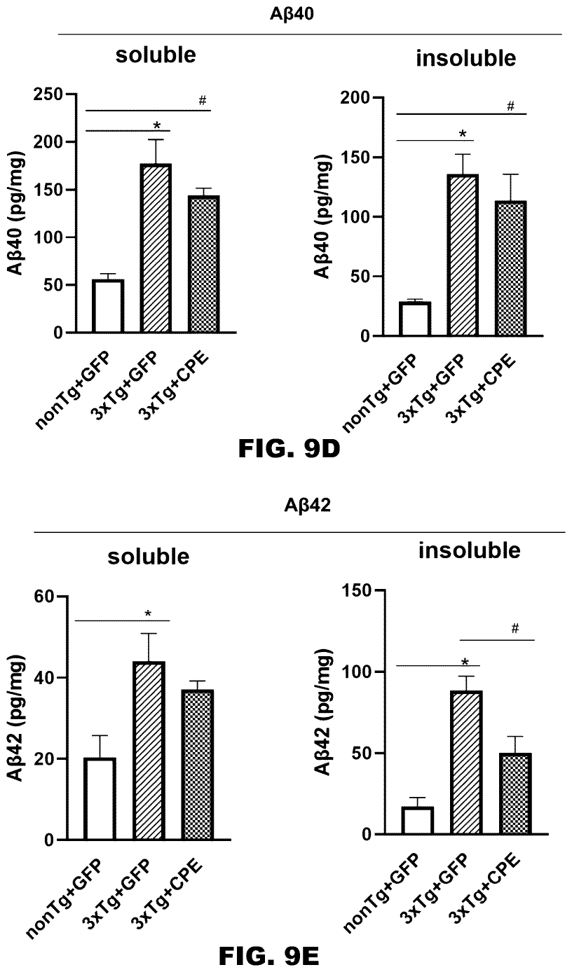

This example demonstrates that CPE minimizes APP expression and Aβ-42 levels in AD. Hyperphosphorylation of tau leading to neurofibrillary formation is a characteristic pathology of AD. FIG. 9A shows increased tau phosphorylation in the 3×Tg-AD compared to non-Tg mice. Hippocampal delivery of AAV-NF-α1/CPE significantly decreased tau hyperphosphorylation in these mice (3×Tg-CPE) to levels comparable to those in non-Tg mice.

Since increased amyloid Aβ42 production and deposition is a hallmark of AD, we examined the expression of APP and Aβ42 levels in 3×Tg-AD mice with and without AAV-NF-α1/CPE treatment. Western blot analysis showed highly elevated levels of APP expression in 3×Tg-AD mice compared to non-Tg mice, which was significantly attenuated in AAV-NF-al/CPE treated 3×Tg-AD mice (FIG. 9B). Morphological studies showed strong APP specific immunostaining in the CA1-3 regions of the hippocampus, and localized in the cell body and neurites of the neurons in 3×Tg-AD mice (FIG. 9C middle panel). However, in 3×Tg-CPE mice, (FIG. 9C right panel), APP immunostaining was greatly attenuated to levels comparable to non-Tg mice (FIG. 9C left panel). Thus AAV-NF-α1/CPE treatment greatly decreased the number of APP positive cells in the hippocampal CA1-3 regions in 3×Tg-CPE mice compared to 3×Tg-GFP mice.

Analysis of APP processed products showed higher levels of both soluble and insoluble A040 in 3×Tg-GFP and 3×Tg-CPE mice compared to non-Tg mice (FIG. 9D). In contrast, while soluble A042 levels were higher in both 3×Tg-GFP and 3×Tg-CPE mice, than non-Tg mice, insoluble A042 was significantly lower in 3×-Tg-CPE mice compared to 3×Tg-GFP mice (FIG. 9E). Thus, AAV-NF-α1/CPE treatment of 3×Tg-AD mice inhibited the up-regulation of APP expression and significantly decreased insoluble A042 production in these mice.

### Example 8

### AAV-Human CPE or AAV-Human CPE-E342Q Gene Delivery in Hippocampus Prevents Learning Impairment and Memory Loss in 3×Tg-AD Mice

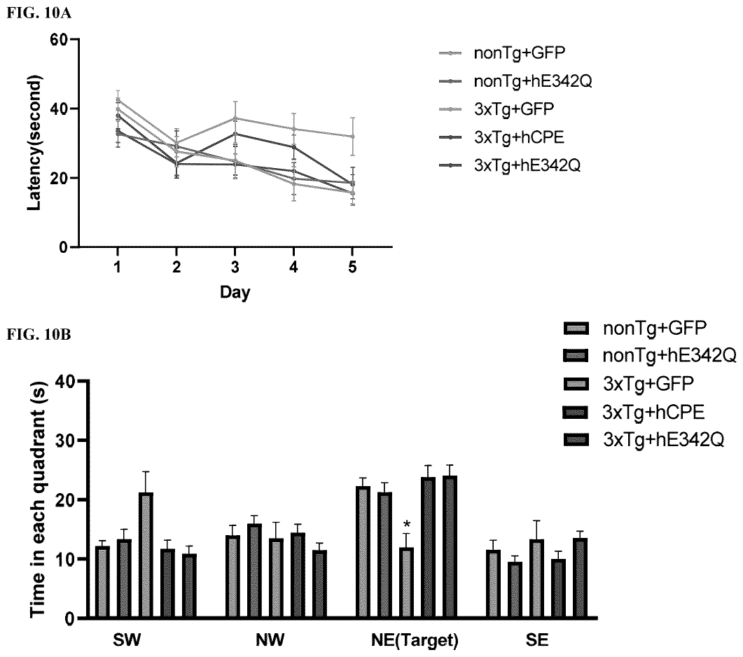

This example demonstrates that human CPE works as well as mouse CPE. In particular, the data establish that human CPE and human CPE-E342Q both prevent learning impairment and memory loss in 3×Tg-AD mice. Thus, the non-enzymatic mutant human CPE-E342Q is effective as human CPE, which in turn is effective as mouse CPE in the examples above. The data are shown in FIGS. 10A-B.

The following sequences are part of the present disclosure.

SEQ ID NO: 1-Mouse wt-CPE CDS (stop codon TAA included)). Bolded nucleotide indicates
where the point mutation occurs for mouse CPE-E342Q mutant.
ATGGCCGGGCGCGGAGGACGGGTGCTGCTGGCGCTGTGTGCCGCGCTGGTGGCCGGCGGGTGGCTGCTGA
CGGCTGAAGCCCAGGAGCCCGGGGCGCCAGCGGCTGGCATGAGGCGCCGCCGGCGGCTCCAGCAAGAGGA
CGGCATCTCCTTCGAGTACCACCGCTATCCAGAGCTGCGCGAGGCGCTGGTGTCCGTATGGCTGCAGTGC
ACCGCCATCAGCAGAATCTACACAGTGGGGCGCAGCTTCGAGGGCCGGGAGCTCCTGGTCATCGAGCTGT
CTGACAACCCCGGGGTCCATGAGCCGGGTGAACCTGAATTTAAATACATTGGGAACATGCATGGTAATGA
GGCGGTTGGACGGGAACTGCTTATTTTCTTGGCCCAGTACCTGTGTAACGAGTACCAGAAAGGCAATGAG
ACAATTGTCAACCTGATCCACAGCACCCGAATTCATATCATGCCCTCCTTGAACCCCGACGGCTTTGAGA
AAGCCGCATCGCAGCCCGGCGAGCTGAAGGACTGGTTTGTGGGCCGCAGCAACGCCCAGGGAATAGATCT
GAACCGTAACTTCCCAGACCTGGACAGGATCGTGTATGTTAATGAGAAAGAAGGCGGTCCTAACAATCAC
CTGCTGAAGAATCTGAAGAAAATTGTGGACCAAAATTCAAAGCTTGCCCCCGAGACCAAGGCTGTCATTC
ACTGGATCATGGACATTCCATTTGTGCTTTCTGCCAATCTGCACGGAGGAGACCTTGTGGCTAATTACCC
ATATGATGAGACACGGAGCGGTACTGCTCACGAATACAGTTCCTGCCCTGATGACGCAATTTTCCAAAGC
TTGGCTCGCGCGTACTCTTCTTTCAACCCAGTCATGTCTGACCCCAATCGACCTCCCTGTCGCAAGAATG
ACGATGACAGCAGCTTTGTAGATGGAACGACCAATGGTGGTGCATGGTACAGCGTCCCCGGTGGAATGCA
AGACTTCAATTACCTGAGCAGCAACTGCTTCGAGATCACTGTGGAGCTTAGCTGTGAGAAGTTCCCACCG
GAAGAGACTCTCAAAAGCTACTGGGAAGATAACAAAAACTCCCTCATCAGCTACCTGGAGCAGATACACC
GAGGTGTTAAAGGGTTTGTCCGTGACCTTCAGGGTAACCCGATTGCCAACGCAACCATCTCTGTGGACGG
GATAGACCATGATGTCACCTCGGCTAAGGATGGGGATTACTGGCGATTGCTTGCTCCTGGAAACTATAAA
CTTACAGCCTCCGCTCCTGGCTACCTGGCAATCACAAAGAAAGTGGCAGTTCCTTTTAGCCCTGCTGTTG
GGGTGGACTTTGAGCTTGAGTCTTTCTCTGAAAGGAAGGAGGAGGAGAAGGAAGAATTGATGGAGTGGTG
GAAAATGATGTCAGAAACTTTGAATTTTTAA
SEQ ID NO: 2-Mouse wt-CPE protein (translation of SEQ ID NO: 1). Bolded and italicized
area includes the pre- (underlined) and pro- (the rest) regions that get processed and
removed to yield the mature protein secreted as the active protein. Pre- and pro- regions
are important in trafficking and folding. Bolded residue E indicates where the mutation
occurs for mouse CPE-E342Q mutant.
MAGRGGRVLLALCAALVAGGWLLTAEAQEPGAPAAGMRRRRRLQQEDGISFEYHRYPELREALVSVWLQC
TAISRIYTVGRSFEGRELLVIELSDNPGVHEPGEPEFKYIGNMHGNEAVGRELLIFLAQYLCNEYQKGNE
TIVNLIHSTRIHIMPSLNPDGFEKAASQPGELKDWFVGRSNAQGIDLNRNFPDLDRIVYVNEKEGGPNNH
LLKNLKKIVDQNSKLAPETKAVIHWIMDIPFVLSANLHGGDLVANYPYDETRSGTAHEYSSCPDDAIFQS
LARAYSSFNPVMSDPNRPPCRKNDDDSSFVDGTTNGGAWYSVPGGMQDENYLSSNCFEITVELSCEKFPP
EETLKSYWEDNKNSLISYLEQIHRGVKGFVRDLQGNPIANATISVDGIDHDVTSAKDGDYWRLLAPGNYK
LTASAPGYLAITKKVAVPFSPAVGVDFELESFSERKEEEKEELMEWWKMMSETLNF
SEQ ID NO: 3-Human wt-CPE (wt-hCPE) CDS (stop codon TAA included). Bolded nucleotide
indicates where the point mutation occurs for human CPE-E342Q mutant.
ATGGCCGGGCGAGGGGGCAGCGCGCTGCTGGCTCTGTGCGGGGCACTGGCTGCCTGCGGGTGGCTCCTGG
GCGCCGAAGCCCAGGAGCCCGGGGCGCCCGCGGCGGGCATGAGGCGGCGCCGGCGGCTGCAGCAAGAGGA
CGGCATCTCCTTCGAGTACCACCGCTACCCCGAGCTGCGCGAGGCGCTCGTGTCCGTGTGGCTGCAGTGC
ACCGCCATCAGCAGGATTTACACGGTGGGGCGCAGCTTCGAGGGCCGGGAGCTCCTGGTCATCGAGCTGT
CCGACAACCCTGGCGTCCATGAGCCTGGTGAGCCTGAATTTAAATACATTGGGAATATGCATGGGAATGA
GGCTGTTGGACGAGAACTGCTCATTTTCTTGGCCCAGTACCTATGCAACGAATACCAGAAGGGGAACGAG
ACAATTGTCAACCTGATCCACAGTACCCGCATTCACATCATGCCTTCCCTGAACCCAGATGGCTTTGAGA
AGGCAGCGTCTCAGCCTGGTGAACTCAAGGACTGGTTTGTGGGTCGAAGCAATGCCCAGGGAATAGATCT
GAACCGGAACTTTCCAGACCTGGATAGGATAGTGTACGTGAATGAGAAAGAAGGTGGTCCAAATAATCAT
CTGTTGAAAAATATGAAGAAAATTGTGGATCAAAACACAAAGCTTGCTCCTGAGACCAAGGCTGTCATTC
ATTGGATTATGGATATTCCTTTTGTGCTTTCTGCCAATCTCCATGGAGGAGACCTTGTGGCCAATTATCC
ATATGATGAGACGCGGAGTGGTAGTGCTCACGAATACAGCTCCTCCCCAGATGACGCCATTTTCCAAAGC
TTGGCCCGGGCATACTCTTCTTTCAACCCGGCCATGTCTGACCCCAATCGGCCACCATGTCGCAAGAATG
ATGATGACAGCAGCTTTGTAGATGGAACCACCAACGGTGGTGCTTGGTACAGCGTACCTGGAGGGATGCA
AGACTTCAATTACCTTAGCAGCAACTGTTTTGAGATCACCGTGGAGCTTAGCTGTGAGAAGTTCCCACCT
GAAGAGACTCTGAAGACCTACTGGGAGGATAACAAAAACTCCCTCATTAGCTACCTTGAGCAGATACACC
GAGGAGTTAAAGGATTTGTCCGAGACCTTCAAGGTAACCCAATTGCGAATGCCACCATCTCCGTGGAAGG
AATAGACCACGATGTTACATCCGCAAAGGATGGTGATTACTGGAGATTGCTTATACCTGGAAACTATAAA
CTTACAGCCTCAGCTCCAGGCTATCTGGCAATAACAAAGAAAGTGGCAGTTCCTTACAGCCCTGCTGCTG
GGGTTGATTTTGAACTGGAGTCATTTTCTGAAAGGAAAGAAGAGGAGAAGGAAGAATTGATGGAATGGTG
GAAAATGATGTCAGAAACTTTAAATTTTTAA
SEQ ID NO: 4-Human wt-CPE protein (translation of SEQ ID NO: 3; 476 aa). Bold and
italicized area includes the pre- (underlined) and pro- (the rest) regions that get
processed and removed to yield the mature protein secreted as the active protein. Pre- and
pro- regions are important in trafficking and folding of the protein in the cell. Bolded
residue E indicates where the mutation occurs for human CPE-E342Q mutant.
MAGRGGSALLALCGALAACGWLLGAEAQEPGAPAAGMRRRRRLQQEDGISFEYHRYPELREALVSVWLQC
TAISRIYTVGRSFEGRELLVIELSDNPGVHEPGEPEFKYIGNMHGNEAVGRELLIFLAQYLCNEYQKGNE
TIVNLIHSTRIHIMPSLNPDGFEKAASQPGELKDWFVGRSNAQGIDLNRNFPDLDRIVYVNEKEGGPNNH
LLKNMKKIVDQNTKLAPETKAVIHWIMDIPFVLSANLHGGDLVANYPYDETRSGSAHEYSSSPDDAIFQS
LARAYSSFNPAMSDPNRPPCRKNDDDSSFVDGTTNGGAWYSVPGGMQDFNYLSSNCFEITVELSCEKFPP
EETLKTYWEDNKNSLISYLEQIHRGVKGFVRDLQGNPIANATISVEGIDHDVTSAKDGDYWRLLIPGNYK
LTASAPGYLAITKKVAVPYSPAAGVDFELESFSERKEEEKEELMEWWKMMSETLNF
SEQ ID NO: 5-Mouse CPE-E342Q CDS (stop codon TAA included)). Bolded nucleotide
represents the point mutation from the wildtype (wt), and the underlined codon translates
to amino acid residue Q at position 342.
ATGGCCGGGCGCGGAGGACGGGTGCTGCTGGCGCTGTGTGCCGCGCTGGTGGCCGGCGGGTGGCTGCTGA
CGGCTGAAGCCCAGGAGCCCGGGGCGCCAGCGGCTGGCATGAGGCGCCGCCGGCGGCTCCAGCAAGAGGA
CGGCATCTCCTTCGAGTACCACCGCTATCCAGAGCTGCGCGAGGCGCTGGTGTCCGTATGGCTGCAGTGC
ACCGCCATCAGCAGAATCTACACAGTGGGGCGCAGCTTCGAGGGCCGGGAGCTCCTGGTCATCGAGCTGT
CTGACAACCCCGGGGTCCATGAGCCGGGTGAACCTGAATTTAAATACATTGGGAACATGCATGGTAATGA
GGCGGTTGGACGGGAACTGCTTATTTTCTTGGCCCAGTACCTGTGTAACGAGTACCAGAAAGGCAATGAG
ACAATTGTCAACCTGATCCACAGCACCCGAATTCATATCATGCCCTCCTTGAACCCCGACGGCTTTGAGA
AAGCCGCATCGCAGCCCGGCGAGCTGAAGGACTGGTTTGTGGGCCGCAGCAACGCCCAGGGAATAGATCT
GAACCGTAACTTCCCAGACCTGGACAGGATCGTGTATGTTAATGAGAAAGAAGGCGGTCCTAACAATCAC
CTGCTGAAGAATCTGAAGAAAATTGTGGACCAAAATTCAAAGCTTGCCCCCGAGACCAAGGCTGTCATTC
ACTGGATCATGGACATTCCATTTGTGCTTTCTGCCAATCTGCACGGAGGAGACCTTGTGGCTAATTACCC
ATATGATGAGACACGGAGCGGTACTGCTCACGAATACAGTTCCTGCCCTGATGACGCAATTTTCCAAAGC
TTGGCTCGCGCGTACTCTTCTTTCAACCCAGTCATGTCTGACCCCAATCGACCTCCCTGTCGCAAGAATG
ACGATGACAGCAGCTTTGTAGATGGAACGACCAATGGTGGTGCATGGTACAGCGTCCCCGGTGGAATGCA
AGACTTCAATTACCTGAGCAGCAACTGCTTCGAGATCACTGTGCAGCTTAGCTGTGAGAAGTTCCCACCG
GAAGAGACTCTCAAAAGCTACTGGGAAGATAACAAAAACTCCCTCATCAGCTACCTGGAGCAGATACACC
GAGGTGTTAAAGGGTTTGTCCGTGACCTTCAGGGTAACCCGATTGCCAACGCAACCATCTCTGTGGACGG
GATAGACCATGATGTCACCTCGGCTAAGGATGGGGATTACTGGCGATTGCTTGCTCCTGGAAACTATAAA
CTTACAGCCTCCGCTCCTGGCTACCTGGCAATCACAAAGAAAGTGGCAGTTCCTTTTAGCCCTGCTGTTG
GGGTGGACTTTGAGCTTGAGTCTTTCTCTGAAAGGAAGGAGGAGGAGAAGGAAGAATTGATGGAGTGGTG
GAAAATGATGTCAGAAACTTTGAATTTTTAA
SEQ ID NO: 6-Mouse CPE-E342Q protein (translation of SEQ ID NO: 5). Bolded residue Q
represents the mutation at position 342.
MAGRGGRVLLALCAALVAGGWLLTAEAQEPGAPAAGMRRRRRLQQEDGISFEYHRYPELREALVSVWLQC
TAISRIYTVGRSFEGRELLVIELSDNPGVHEPGEPEFKYIGNMHGNEAVGRELLIFLAQYLCNEYQKGNE
TIVNLIHSTRIHIMPSLNPDGFEKAASQPGELKDWFVGRSNAQGIDLNRNFPDLDRIVYVNEKEGGPNNH
LLKNLKKIVDQNSKLAPETKAVIHWIMDIPFVLSANLHGGDLVANYPYDETRSGTAHEYSSCPDDAIFQS
LARAYSSFNPVMSDPNRPPCRKNDDDSSFVDGTTNGGAWYSVPGGMQDFNYLSSNCFEITVQLSCEKFPP
EETLKSYWEDNKNSLISYLEQIHRGVKGFVRDLQGNPIANATISVDGIDHDVTSAKDGDYWRLLAPGNYK
LTASAPGYLAITKKVAVPFSPAVGVDFELESFSERKEEEKEELMEWWKMMSETLNF
SEQ ID NO: 7-Human CPE-E342Q (hCPE-E342Q) CDS (stop codon TAA included). Bolded
nucleotide represents the point mutation from the wildtype (wt), and the underlined codon
translates to amino acid residue Q at position 342.
ATGGCCGGGCGAGGGGGCAGCGCGCTGCTGGCTCTGTGCGGGGCACTGGCTGCCTGCGGGTGGCTCCTGG
GCGCCGAAGCCCAGGAGCCCGGGGCGCCCGCGGCGGGCATGAGGCGGCGCCGGCGGCTGCAGCAAGAGGA
CGGCATCTCCTTCGAGTACCACCGCTACCCCGAGCTGCGCGAGGCGCTCGTGTCCGTGTGGCTGCAGTGC
ACCGCCATCAGCAGGATTTACACGGTGGGGCGCAGCTTCGAGGGCCGGGAGCTCCTGGTCATCGAGCTGT
CCGACAACCCTGGCGTCCATGAGCCTGGTGAGCCTGAATTTAAATACATTGGGAATATGCATGGGAATGA
GGCTGTTGGACGAGAACTGCTCATTTTCTTGGCCCAGTACCTATGCAACGAATACCAGAAGGGGAACGAG
ACAATTGTCAACCTGATCCACAGTACCCGCATTCACATCATGCCTTCCCTGAACCCAGATGGCTTTGAGA
AGGCAGCGTCTCAGCCTGGTGAACTCAAGGACTGGTTTGTGGGTCGAAGCAATGCCCAGGGAATAGATCT
GAACCGGAACTTTCCAGACCTGGATAGGATAGTGTACGTGAATGAGAAAGAAGGTGGTCCAAATAATCAT
CTGTTGAAAAATATGAAGAAAATTGTGGATCAAAACACAAAGCTTGCTCCTGAGACCAAGGCTGTCATTC
ATTGGATTATGGATATTCCTTTTGTGCTTTCTGCCAATCTCCATGGAGGAGACCTTGTGGCCAATTATCC
ATATGATGAGACGCGGAGTGGTAGTGCTCACGAATACAGCTCCTCCCCAGATGACGCCATTTTCCAAAGC
TTGGCCCGGGCATACTCTTCTTTCAACCCGGCCATGTCTGACCCCAATCGGCCACCATGTCGCAAGAATG
ATGATGACAGCAGCTTTGTAGATGGAACCACCAACGGTGGTGCTTGGTACAGCGTACCTGGAGGGATGCA
AGACTTCAATTACCTTAGCAGCAACTGTTTTGAGATCACCGTGCAGCTTAGCTGTGAGAAGTTCCCACCT
GAAGAGACTCTGAAGACCTACTGGGAGGATAACAAAAACTCCCTCATTAGCTACCTTGAGCAGATACACC
GAGGAGTTAAAGGATTTGTCCGAGACCTTCAAGGTAACCCAATTGCGAATGCCACCATCTCCGTGGAAGG
AATAGACCACGATGTTACATCCGCAAAGGATGGTGATTACTGGAGATTGCTTATACCTGGAAACTATAAA
CTTACAGCCTCAGCTCCAGGCTATCTGGCAATAACAAAGAAAGTGGCAGTTCCTTACAGCCCTGCTGCTG
GGGTTGATTTTGAACTGGAGTCATTTTCTGAAAGGAAAGAAGAGGAGAAGGAAGAATTGATGGAATGGTG
GAAAATGATGTCAGAAACTTTAAATTTTTAA
SEQ ID NO: 8-Human CPE-E342Q (hCPE-E342Q) protein (translation of SEQ ID NO: 7).
Bolded residue Q represents the mutation at position 342.
MAGRGGSALLALCGALAACGWLLGAEAQEPGAPAAGMRRRRRLQQEDGISFEYHRYPELREALVSVWLQC
TAISRIYTVGRSFEGRELLVIELSDNPGVHEPGEPEFKYIGNMHGNEAVGRELLIFLAQYLCNEYQKGNE
TIVNLIHSTRIHIMPSLNPDGFEKAASQPGELKDWFVGRSNAQGIDLNRNFPDLDRIVYVNEKEGGPNNH
LLKNMKKIVDQNTKLAPETKAVIHWIMDIPFVLSANLHGGDLVANYPYDETRSGSAHEYSSSPDDAIFQS
LARAYSSFNPAMSDPNRPPCRKNDDDSSFVDGTTNGGAWYSVPGGMQDENYLSSNCFQITVELSCEKFPP
EETLKTYWEDNKNSLISYLEQIHRGVKGFVRDLQGNPIANATISVEGIDHDVTSAKDGDYWRLLIPGNYK
LTASAPGYLAITKKVAVPYSPAAGVDFELESFSERKEEEKEELMEWWKMMSETLNF
SEQ ID NO: 9-Mouse CPE-ΔN mRNA
GTCCATGAGCCGGGTGAACCTGAATTTAAATACATTGGGAACATGCATGGTAATGAGGCGGTTGGACGGGAACTGCTTATTTTCTT
GGCCCAGTACCTGTGTAACGAGTACCAGAAAGGCAATGAGACAATTGTCAACCTGATCCACAGCACCCGAATTCATATCATGCCCT
CCTTGAACCCCGACGGCTTTGAGAAAGCCGCATCGCAGCCCGGCGAGCTGAAGGACTGGTTTGTGGGCCGCAGCAACGCCCAGGGA
ATAGATCTGAACCGTAACTTCCCAGACCTGGACAGGATCGTGTATGTTAATGAGAAAGAAGGCGGTCCTAACAATCACCTGCTGAA
GAATCTGAAGAAAATTGTGGACCAAAATTCAAAGCTTGCCCCCGAGACCAAGGCTGTCATTCACTGGATCATGGACATTCCATTTG
TGCTTTCTGCCAATCTGCACGGAGGAGACCTTGTGGCTAATTACCCATATGATGAGACACGGAGCGGTACTGCTCACGAATACAGT
TCCTGCCCTGATGACGCAATTTTCCAAAGCTTGGCTCGCGCGTACTCTTCTTTCAACCCAGTCATGTCTGACCCCAATCGACCTCC
CTGTCGCAAGAATGACGATGACAGCAGCTTTGTAGATGGAACGACCAATGGTGGTGCATGGTACAGCGTCCCCGGTGGAATGCAAG
ACTTCAATTACCTGAGCAGCAACTGCTTCGAGATCACTGTGGAGCTTAGCTGTGAGAAGTTCCCACCGGAAGAGACTCTCAAAAGC
TACTGGGAAGATAACAAAAACTCCCTCATCAGCTACCTGGAGCAGATACACCGAGGTGTTAAAGGGTTTGTCCGTGACCTTCAGGG
TAACCCGATTGCCAACGCAACCATCTCTGTGGACGGGATAGACCATGATGTCACCTCGGCTAAGGATGGGGATTACTGGCGATTGC
TTGCTCCTGGAAACTATAAACTTACAGCCTCCGCTCCTGGCTACCTGGCAATCACAAAGAAAGTGGCAGTTCCTTTTAGCCCTGCT
GTTGGGGTGGACTTTGAGCTTGAGTCTTTCTCTGAAAGGAAGGAGGAGGAGAAGGAAGAATTGATGGAGTGGTGGAAAATGATGTC
AGAAACTTTGAATTTTTAAGAAAGGCTTCTAACTAATTGCTTTAATCTATCTATAGACTGTAGTAAGATGCAATGTGGCTCTTTTC
TTTTAGGTTGTGTGCAGTTGATATTTAACATTGATTTATTTTTGATCATTTAAGTAATAGTTAGTAATCACGTAAATACACCCGGA
CAGAAATATAATGTCTGGATCTACTTCATTCTTACATCAACATTCACTTTAAAATCTATCGAAGCTCTTTTAACGTAATGGGTGAC
AATGTCACATGACAGATGCCATGAAGAAGTCAACCGATATAGCTTGGATCTGTGAACCCTGTACTGCGAGAATCACATAGTTCCAT
ATAAGTTGTCCTTAGTCTCTTGTGCTGATTCACTGTATAAGCATGATCCTGGTAATGCACTTTGGATGGGAAGAAAATGTACGTGC
TTTTCAGAGGGGCTCTGAACAGAATGAAAACCTAGTTCTTGCGTGTACTTTGAAGAATGGAATTGTATTAGTCAGCCTGTTAATGC
CACTTCAGAGTTTGGGGTTTTGTCTTGATTGTAGATTGGCCCAGAATTGCATTCTGATGAATAAAGGCAAAAAAAAAAAAAAAAAA
AAAAAAAAA
SEQ ID NO: 10-Mouse CPE-ΔN CDS
ATGCATGGTAATGAGGCGGTTGGACGGGAACTGCTTATTTTCTTGGCCCAGTACCTGTGTAACGAGTACCAGAAAGGCAATGAGAC
AATTGTCAACCTGATCCACAGCACCCGAATTCATATCATGCCCTCCTTGAACCCCGACGGCTTTGAGAAAGCCGCATCGCAGCCCG
GCGAGCTGAAGGACTGGTTTGTGGGCCGCAGCAACGCCCAGGGAATAGATCTGAACCGTAACTTCCCAGACCTGGACAGGATCGTG
TATGTTAATGAGAAAGAAGGCGGTCCTAACAATCACCTGCTGAAGAATCTGAAGAAAATTGTGGACCAAAATTCAAAGCTTGCCCC
CGAGACCAAGGCTGTCATTCACTGGATCATGGACATTCCATTTGTGCTTTCTGCCAATCTGCACGGAGGAGACCTTGTGGCTAATT
ACCCATATGATGAGACACGGAGCGGTACTGCTCACGAATACAGTTCCTGCCCTGATGACGCAATTTTCCAAAGCTTGGCTCGCGCG
TACTCTTCTTTCAACCCAGTCATGTCTGACCCCAATCGACCTCCCTGTCGCAAGAATGACGATGACAGCAGCTTTGTAGATGGAAC
GACCAATGGTGGTGCATGGTACAGCGTCCCCGGTGGAATGCAAGACTTCAATTACCTGAGCAGCAACTGCTTCGAGATCACTGTGG
AGCTTAGCTGTGAGAAGTTCCCACCGGAAGAGACTCTCAAAAGCTACTGGGAAGATAACAAAAACTCCCTCATCAGCTACCTGGAG
CAGATACACCGAGGTGTTAAAGGGTTTGTCCGTGACCTTCAGGGTAACCCGATTGCCAACGCAACCATCTCTGTGGACGGGATAGA
CCATGATGTCACCTCGGCTAAGGATGGGGATTACTGGCGATTGCTTGCTCCTGGAAACTATAAACTTACAGCCTCCGCTCCTGGCT
ACCTGGCAATCACAAAGAAAGTGGCAGTTCCTTTTAGCCCTGCTGTTGGGGTGGACTTTGAGCTTGAGTCTTTCTCTGAAAGGAAG
GAGGAGGAGAAGGAAGAATTGATGGAGTGGTGGAAAATGATGTCAGAAACTTTGAATTTTTAA
SEQ ID NO: 11-Mouse CPE-ΔN protein (translation of SEQ ID NO: 10)
MHGNEAVGRELLIFLAQYLCNEYQKGNETIVNLIHSTRIHIMPSLNPDGFEKAASQPGELKDWFVGRSNAQGIDLNR
NFPDLDRIVYVNEKEGGPNNHLLKNLKKIVDQNSKLAPETKAVIHWIMDIPFVLSANLHGGDLVANYPYDETRSGTA
HEYSSCPDDAIFQSLARAYSSFNPVMSDPNRPPCRKNDDDSSFVDGTTNGGAWYSVPGGMQDFNYLSSNCFEITVEL
SCEKFPPEETLKSYWEDNKNSLISYLEQIHRGVKGFVRDLQGNPIANATISVDGIDHDVTSAKDGDYWRLLAPGNYK
LTASAPGYLAITKKVAVPFSPAVGVDFELESFSERKEEEKEELMEWWKMMSETLNF
SEQ ID NO: 12-Human CPE-AN mRNA

| | |
|---|---|
| 1 | CATTCAGCCG GGGAAGGTGA GGCGAGTAGA GGCTGGTGCG GAACTTGCCG CCCCCTGAGG |
| 61 | CGGCGCCGGC GGCTGCAGCA AGAGGACGGC ATCTCCTTCG AGTACCACCG CTACCCCGAG |
| 121 | CTGCGCGAGG CGCTCGTGTC CGTGTGGCTG CAGTGCACCG CCATCAGCAG GATTTACACG |
| 181 | GTGGGGCGCA GCTTCGAGGG CCGGGAGCTC CTGGTCATCG AGCTGTCCGA CAACCCTGGC |
| 241 | GTCCATGAGC CTGGTGAGCC TGAATTTAAA TACATTGGGA ATATGCATGG GAATGAGGCT |
| 301 | GTTGGACGAG AACTGCTCAT TTTCTTGGCC CAGTACCTAT GCAACGAATA CCAGAAGGGG |
| 361 | AACGAGACAA TTGTCAACCT GATCCACAGT ACCCGCATTC ACATCATGCC TTCCCTGAAC |
| 421 | CCAGATGGCT TTGAGAAGGC AGCGTCTCAG CCTGGTGAAC TCAAGGACTG GTTTGTGGGT |
| 481 | CGAAGCAATG CCCAGGGAAT AGATCTGAAC CGGAACTTTC CAGACCTGGA TAGGATAGTG |
| 541 | TACGTGAATG AGAAAGAAGG TGGTCCAAAT AATCATCTGT TGAAAAATAT GAAGAAAATT |
| 601 | GTGGATCAAA ACACAAAGCT TGCTCCTGAG ACCAAGGCTG TCATTCATTG GATTATGGAT |
| 661 | ATTCCTTTTG TGCTTTCTGC CAATCTCCAT GGAGGAGACC TTGTGGCCAA TTATCCATAT |
| 721 | GATGAGACGC GGAGTGGTAG TGCTCACGAA TACAGCTCCT CCCCAGATGA CGCCATTTTC |
| 781 | CAAAGCTTGG CCCGGGCATA CTCTTCTTTC AACCCGGCCA TGTCTGACCC CAATCGGCCA |
| 841 | CCATGTCGCA AGAATGATGA TGACAGCAGC TTTGTAGATG GAACCACCAA CGGTGGTGCT |
| 901 | TGGTACAGCG TACCTGGAGG GATGCAAGAC TTCAATTACC TTAGCAGCAA CTGTTTTGAG |
| 961 | ATCACCGTGG AGCTTAGCTG TGAGAAGTTC CCACCTGAAG AGACTCTGAA GACCTACTGG |
| 1021 | GAGGATAACA AAAACTCCCT CATTAGCTAC CTTGAGCAGA TACACCGAGG AGTTAAAGGA |
| 1081 | TTTGTCCGAG ACCTTCAAGG TAACCCAATT GCGAATGCCA CCATCTCCGT GGAAGGAATA |
| 1141 | GACCACGATG TTACATCCGC AAAGGATGGT GATTACTGGA GATTGCTTAT ACCTGGAAAC |
| 1201 | TATAAACTTA CAGCCTCAGC TCCAGGCTAT CTGGCAATAA CAAAGAAAGT GGCAGTTCCT |
| 1261 | TACAGCCCTG CTGCTGGGGT TGATTTTGAA CTGGAGTCAT TTTCTGAAAG GAAAGAAGAG |
| 1321 | GAGAAGGAAG AATTGATGGA ATGGTGGAAA ATGATGTCAG AAACTTTAAA TTTTTAAAAA |
| 1381 | GGCTTCTAGT TAGCTGCTTT AAATCTATCT ATATAATGTA GTATGATGTA ATGTGGTCTT |
| 1441 | TTTTTTAGAT TTTGTGCAGT TAATACTTAA CATTGATTTA TTTTTTAATC ATTTAAATAT |
| 1501 | TAATCAACTT TCCTTAAAAT AAATAGCCTC TTAGGTAAAA AAAAAAAAAA AAAAAAAAAA |
| 1561 | AAAAAAAAA |

SEQ ID NO: 13-Human CPE-ΔN CDS
ATGCATGGGAATGAGGCTGTTGGACGAGAACTGCTCATTTTCTTGGCCCAGTACCTATGCAACGAATACCAGAAGGGGAACGAGAC
AATTGTCAACCTGATCCACAGTACCCGCATTCACATCATGCCTTCCCTGAACCCAGATGGCTTTGAGAAGGCAGCGTCTCAGCCTG
GTGAACTCAAGGACTGGTTTGTGGGTCGAAGCAATGCCCAGGGAATAGATCTGAACCGGAACTTTCCAGACCTGGATAGGATAGTG
TACGTGAATGAGAAAGAAGGTGGTCCAAATAATCATCTGTTGAAAAATATGAAGAAAATTGTGGATCAAAACACAAAGCTTGCTCC
TGAGACCAAGGCTGTCATTCATTGGATTATGGATATTCCTTTTGTGCTTTCTGCCAATCTCCATGGAGGAGACCTTGTGGCCAATT
ATCCATATGATGAGACGCGGAGTGGTAGTGCTCACGAATACAGCTCCTCCCCAGATGACGCCATTTTCCAAAGCTTGGCCCGGGCA
TACTCTTCTTTCAACCCGGCCATGTCTGACCCCAATCGGCCACCATGTCGCAAGAATGATGATGACAGCAGCTTTGTAGATGGAAC
CACCAACGGTGGTGCTTGGTACAGCGTACCTGGAGGGATGCAAGACTTCAATTACCTTAGCAGCAACTGTTTTGAGATCACCGTGG
AGCTTAGCTGTGAGAAGTTCCCACCTGAAGAGACTCTGAAGACCTACTGGGAGGATAACAAAAACTCCCTCATTAGCTACCTTGAG
CAGATACACCGAGGAGTTAAAGGATTTGTCCGAGACCTTCAAGGTAACCCAATTGCGAATGCCACCATCTCCGTGGAAGGAATAGA
CCACGATGTTACATCCGCAAAGGATGGTGATTACTGGAGATTGCTTATACCTGGAAACTATAAACTTACAGCCTCAGCTCCAGGCT
ATCTGGCAATAACAAAGAAAGTGGCAGTTCCTTACAGCCCTGCTGCTGGGGTTGATTTTGAACTGGAGTCATTTTCTGAAAGGAAA
GAAGAGGAGAAGGAAGAATTGATGGAATGGTGGAAAATGATGTCAGAAACTTTAAATTTTTAA
SEQ ID NO: 14-Human CPE-AN protein (translation of SEQ ID NO: 13)
MHGNEAVGRELLIFLAQYLCNEYQKGNETIVNLIHSTRIHIMPSLNPDGFEKAASQPGELKDWFVGRSNAQGIDLNR
NFPDLDRIVYVNEKEGGPNNHLLKNMKKIVDQNTKLAPETKAVIHWIMDIPFVLSANLHGGDLVANYPYDETRSGSA
HEYSSSPDDAIFQSLARAYSSFNPAMSDPNRPPCRKNDDDSSFVDGTTNGGAWYSVPGGMQDFNYLSSNCFEITVEL
SCEKFPPEETLKTYWEDNKNSLISYLEQIHRGVKGFVRDLQGNPIANATISVEGIDHDVTSAKDGDYWRLLIPGNYK
LTASAPGYLAITKKVAVPYSPAAGVDFELESFSERKEEEKEELMEWWKMMSET

1. A method of treating a neurodegenerative disease or preventing onset or progression of a neurodegenerative disease in a subject in need thereof, comprising administering a mouse or human Carboxypeptidase E (CPE) protein to the subject, wherein the CPE protein comprises the amino acid sequence set forth in SEQ ID NO: 2, 4, 6, 8, 11, or 14; and wherein the neurodegenerative disease is selected from the group consisting of Alzheimer's disease (AD), Parkinson's disease (PD), dementia, frontotemporal dementia (FTD), depression, bipolar disorder, amyotrophic lateral sclerosis (ALS), spinal cord injury, traumatic brain injury (TBI), stroke, ischemia, and Down's syndrome.

2. (canceled)

3. (canceled)

4. The method of claim 1, wherein the CPE protein comprises the amino acid sequence set forth in SEQ ID NO:2 or SEQ ID NO:4.

5. The method of claim 21, wherein the CPE protein is CPE-E342Q comprising the amino acid sequence set forth in SEQ ID NO:6 or SEQ ID NO:8.

6. The method of claim 21, wherein the CPE protein is a N-terminal-truncated variant (CPE-ΔN), wherein the amino acid sequence of the CPE protein consists of the sequence set forth in SEQ ID NO:11 or SEQ ID NO:14.

7. The method of claim 1, wherein the CPE protein is encoded by a CPE polynucleotide.

8. The method of claim 7, wherein the CPE polynucleotide comprises a nucleic acid sequence at least 95% identical to the sequence set forth in SEQ ID NO:1 or SEQ ID NO:3.

9. The method of claim 8, wherein the CPE polynucleotide comprises the nucleic acid sequence set forth in SEQ ID NO:1 or SEQ ID NO:3.

10. The method of claim 7, wherein the CPE polynucleotide comprises the nucleic acid sequence set forth in SEQ ID NO:5 or SEQ ID NO:7.

11. The method of claim 7, wherein the CPE polynucleotide comprises the nucleic acid sequence set forth in SEQ ID NO:10 or SEQ ID NO:13.

12. The method of claim 7, wherein the CPE polynucleotide is contained in an expression vector.

13. The method of claim 12, wherein the expression vector is an adeno-associated virus (AAV) construct.

14. The method of claim 13, wherein the AAV construct is a AAV1/2 hybrid construct or an AAV9 construct.

15. (canceled)

16. The method of claim 7, wherein the CPE polynucleotide is contained in a pharmaceutical composition.

17. The method of claim 16, wherein the pharmaceutical composition is administered via injection into the brain of the subject.

18. The method of claim 17, wherein the pharmaceutical composition is administered via injection into the hippocampus of the subject.

19. The method of claim 16, wherein the pharmaceutical composition is administered via nasal spray to the subject.

20. The method of claim 16, wherein the pharmaceutical composition comprises an extracellular vesicle encapsulating the CPE protein or CPE polynucleotide.

21. The method of claim 20, wherein the pharmaceutical composition is administered into the cerebrospinal fluid of the subject.

22. The method of claim 16, wherein the pharmaceutical composition is administered at a dose effective to produce about 40% to about 100% increased level of CPE in the neurons of the subject, as compared to an untreated subject.

23.-44. (canceled)
